(19) United States
(12) Patent Application Publication
(10) Pub. No.: US 2008/0183128 A1
(43) Pub. Date: Jul. 31, 2008

(54) Methods, devices and systems for treatment and/or diagnosis of disorders of the ear, nose and throat

(76) Inventors: John Morriss, Portolla Valley, CA (US); Mei Pader, Fremont, CA (US); Joshua Makower, Los Altos, CA (US)

Correspondence Address: STEPTOE & JOHNSON - ACCLARENT, INC., 2121 AVENUE OF THE STARS, SUITE 2800, LOS ANGELES, CA, 90067, US

(21) Appl. No.: 12/011,100

(22) Filed: Jan. 23, 2008

Related U.S. Application Data
(60) Provisional application No. 60/897,446, filed on Jan. 24, 2007.

(51) Int. Cl. A61M 3/02; A61M 25/09; A61M 25/00

(52) U.S. Cl. 604/35; 604/27; 604/164.13; 604/523

(57) ABSTRACT

A method for irrigating a paranasal sinus may involve advancing a distal portion of a sinus irrigation catheter through an ostium of a paranasal sinus into the sinus and passing fluid through multiple ports disposed at different locations along a length of the catheter distal portion into the sinus. A flexible irrigation catheter device for irrigating a paranasal sinus may include: an elongate catheter body having a proximal end, a distal end, a lumen therebetween, and a tapered distal portion extending proximally from the distal end of the catheter body; a proximal hub coupled with the proximal end of the catheter body for connecting to a source of irrigation fluid; multiple side ports disposed along the catheter body closer to the distal end than the proximal end and in fluid communication with the lumen; and a distal end port at the distal end of the catheter body.

This claims priority to U.S. Provisional Patent Application Ser. No. 60/897,446 (Attorney Docket No. ACCL-007CIPPRV), entitled “Methods, Devices and Systems for Treatment and/or Diagnosis of Disorders of the Ear, Nose and Throat,” filed Jan. 24, 2007, the full disclosure of which is hereby incorporated by reference.

This application hereby incorporates each of the following documents herein, in their entireties, by reference thereto: U.S. application Ser. No. 11/193,020, filed Jul. 29, 2005; U.S. application Ser. No. 10/829,917 filed on Apr. 21, 2004; U.S. Patent Application Publication No. 2005/0240147; U.S. application Ser. No. 10/944,270 filed on Sep. 17, 2004; U.S. Patent Application Publication No. 2006/0004323; U.S. application Ser. No. 11/116,118 filed on Apr. 26, 2005; U.S. Patent Application Publication No. 2006/0004286; U.S. application Ser. No. 11/150,847 filed on Jun. 10, 2005; U.S. Patent Application Publication No. 2006/0210605; U.S. application Ser. No. 11/037,548 filed on Jan. 18, 2005; U.S. application Ser. No. 10/912,578 filed on Aug. 4, 2004; U.S. Patent Application Publication No. 2005/0245906; U.S. application Ser. No. 11/522,497, filed Sep. 15, 2006; and U.S. Provisional Application No. 60/844,874, filed Sep. 15, 2006.

### BACKGROUND OF THE INVENTION

Surgical treatments for sinusitis and other disorders of the ear, nose and throat have evolved slowly over the years. In current clinical practice, functional endoscopic sinus surgery (FESS) is often used to treat sinusitis or other disorders where drainage of mucous is impaired and/or chronic infections are present. In FESS, an endoscope is inserted into the nose and, under visualization through the endoscope, the surgeon may remove diseased or hypertrophic tissue or bone and may enlarge the ostia of the sinuses to restore normal drainage of the sinuses. FESS procedures can be effective in the treatment of sinusitis and for the removal of tumors, polyps and other aberrant growths from the nose. Other endoscopic intranasal procedures have been used to remove pituitary tumors, to treat Graves disease (i.e., a complication of hyperthyroidism which results in protrusion of the eyes) and surgical repair of rare conditions wherein cerebrospinal fluid leaks into the nose (i.e., cerebrospinal fluid rhinorrhea).

The surgical instruments used in the prior art FESS procedures having included applicators, chisels, curettes, elevators, forceps, gouges, hooks, knives, saws, mallets, morselizers, needle holders, osteotomes, ostium seekers, probes, punches, backbiters, rasps, retractors, rongeurs, scissors, snares, specula, suction cannulae and trocars. The majority of such instruments are of substantially rigid design.

Although FESS continues to be the gold standard therapy for severe sinuses, it has several shortfalls. Often patients complain of the post-operative pain and bleeding associated with the procedure, and a significant subset of patients remain symptomatic even after multiple surgeries. Since FESS is considered an option only for the most severe cases (those showing abnormalities under CT scan), a large population of patients exist that can neither tolerate the prescribed medications nor be considered candidates for surgery. Further, because the methodologies to assess sinus disease are primarily static measurements (CT, MRI), patients whose symptoms are episodic are often simply offered drug therapy when in fact underlying mechanical factors may play a significant role. This leaves a large population of patients in need of relief, unwilling or afraid to take steroids, but not sick enough to qualify for FESS surgery.

Some experimental or investigational procedures have also been performed in an effort to treat sinusitis by methods that are less invasive and/or less damaging to ancillary tissues than FESS: For example, European physicians have reported the use of a hydrophilic guidewire and standard PTCA balloon catheter to treat restenosis of surgically created openings in diseased frontal sinuses and stenotic nasal conae. Gottmann, D., Strohm, M., Strecker, E. P., Karlsruhe, D. E., Balloon dilatation of Recurrent Ostial Oclusion of the Frontal Sinus, Abstract No. B-0453, European Congress of Radiology (2001); Strohm, M., Gottmann, D., Treatment of Stenoses of Upper Air Routes by Balloon Dilation, Proceeding of the 83.sup.rd Annual Convention of the Association of West German ENT Physicians (1999). The interventions described in this abstract were conducted only on frontal sinuses that had previously been surgically modified and nasal conae. These techniques were not reported to be useable for the treatment of sinus ostia that has not previously been surgically altered or ostia of sinuses other than the easily accessible frontal sinuses. Also, in these reported cases, standard vascular guidewires and angioplasty balloon catheters were used. The techniques described in these publications have not been widely adopted by ENT surgeons, possibly due to the fact that they lacked important novel improvements and modifications as described in this patent application and prior U.S. patent application Ser. Nos. 10/829,917; 10/912,578; 10/829,917; 10/944,270; 11/116,118; 11/150,847; 11/193,020 and 11/037,548, of which this application is a continuation-in-part.

Other methods and devices for sinus intervention using dilating balloons have been disclosed in U.S. Pat. No. 2,525,183 (Robison) and U.S. Patent Publication No. 2004/0064150 A1 (Becker). For example, U.S. Pat. No. 2,525,183 (Robison) discloses an inflatable pressure device which can be inserted following sinus surgery and inflated within the sinus. The patent does not disclose device designs and methods for flexibly navigating through the complex nasal anatomy to access the natural ostia of the sinuses. The discussion of balloon materials is also fairly limited to thin flexible materials like rubber which are most likely to be inadequate for dilating the bony ostia of the sinus.

U.S. patent publication No. 2004/0064150 A1 (Becker) discloses balloon catheters formed of a stiff hypotube to be pushed into a sinus. The balloon catheters have a stiff hypotube with a fixed pre-set angle that enables them to be pushed into the sinus. In at least some procedures wherein it is desired to position the balloon catheter in the ostium of a paranasal sinus, it is necessary to advance the balloon catheter through complicated or tortuous anatomy in order to properly position the balloon catheter within the desired sinus ostium. Also, there is a degree of individual variation in the intranasal and paranasal anatomy of human beings, thus making it difficult to design a stiff-shaft balloon catheter that is optimally shaped for use in all individuals. Indeed, rigid catheters formed of hypotubes that have pre-set angles cannot be easily adjusted by the physician to different shapes to account for individual variations in the anatomy. In view of this, the Becker patent application describes the necessity of having available a set of balloon catheters, each having a particular fixed angle so that the physician can select the appropriate catheter for the patient's anatomy. The requirement to test multiple disposable catheters for fit is likely to be very expensive and impractical. Moreover, if such catheter are disposable items (e.g., not sterilizable and reusable) the need to test and discard a number of catheters before finding one that has the ideal bend angle even further exacerbates the expense factor of Becker's approach.

More recently, new devices, systems and methods have been devised to enable the performance of FESS procedures and other ENT surgeries with minimal or no removal or modification of normal anatomical structures. Such new methods include, but are not limited to, uncinate-sparing procedures using Balloon Sinuplasty™ tools and uncinate-sparing ethmoidectomy procedures using catheters, non-rigid instruments and advanced imaging techniques (Acclarent, Inc., Menlo Park, Calif.). Examples of these new devices, systems and methods are described in incorporated U.S. patent application Ser. No. 10/829,917 entitled Devices, Systems and Methods for Diagnosing and Treating Sinusitis and Other Disorders of the Ears, Nose and/or Throat; Ser. No. 10/944,270 entitled Apparatus and Methods for Dilating and Modifying Ostia of Paranasal Sinuses and Other Intranasal or Paranasal Structures; Ser. No. 11/116,118 entitled Methods and Devices for Performing Procedures Within the Ear, Nose, Throat and Paranasal Sinuses filed Apr. 26, 2005 and Ser. No. 11/150,847 filed Jun. 10, 2005, each of which is hereby incorporated herein, in its entirety. Procedures using Balloon Sinuplasty™ tools such as those described in the above-noted applications, for example, are performable using various types of guidance including but not limited to C-arm fluoroscopy, transnasal endoscopy, optical image guidance and/or electromagnetic image guidance.

Lavage or irrigation procedures have been performed with a straight, flexible tube that is advanceable to some regions of the anatomy to deliver irrigatino or suction to the region from an opening in a distal end of the tube. Problems with these tube and procedures have included kinking of the tube when passed through a guide catether having a bend in the distal end portion adapted to bend the tube toward a particular anatomical location, poor tracking over a guidewire to deliver a distal end of the tube to a desired location, possibly due to stiffness (insufficient flexibility) of the tube and/or tolerance between the lumen of the tube and the guidewire over which it is tracking being too great, tube diameter too large for placement in some locations, and tube diameter too large to be used with small guide catheters (having a relatively small inside diameter). Further the ability to completely rinse out an area such as a sinus has sometimes been compromised, by any of the drawbacks mentioned above and/or ineffective spray delivered from the tube.

There is a continuing need for devices, systems and methods that are optimal for minimally invasive treatment of sinusitis and other ear, nose and throat disorders.

### BRIEF DESCRIPTION OF THE DRAWINGS

FIG. 1 shows components that are useable together in an irrigation system according to one embodiment of the present application.

FIG. 2A is a partial view of an irrigation catheter according to an embodiment of the present invention.

FIG. 2B is a longitudinal sectional view of FIG. 2A.

FIG. 2C is an enlarged view of the portion of FIG. 2A within circle 2C.

FIG. 2D is an enlarged view of the portion of FIG. 2B within circle 2D.

FIG. 3A is a partial view of an irrigation system showing a distal tip portion of a stylet extending distally from a distal end of an irrigation catheter.

FIG. 3B illustrates an irrigation system having been inserted though a guide catheter.

FIG. 3C is a cross-sectional view of a distal tip portion of a stylet.

FIG. 3D is a cross-sectional view of an intermediate portion of a stylet.

FIG. 3E is a cross-sectional view of a proximal portion of a stylet.

FIG. 3F illustrates a distal tip portion of a stylet having been shaped to set a bend to facilitate steering.

FIG. 3G is a longitudinal sectional view of a distal tip portion of a stylet and a distal end portion of an irrigation catheter.

FIG. 4A is a partial, longitudinal sectional view of a removable stylet.

FIG. 4B is an enlarged view of the portion of FIG. 4A within circle 4B.

FIG. 4C is an enlarged view of the portion of FIG. 4A within circle 4C.

FIG. 5A is a partial view of an irrigation system comprising an irrigation catheter having an integrated distal tip portion of a stylet.

FIGS. 5B and 5C are opposite side view of another irrigation system comprising an irrigation catheter having an integrated distal tip portion of a stylet.

FIG. 6A illustrates a portion of a structurally reinforced tubing.

FIG. 6B illustrates a portion of an irrigation system having an irrigation catheter with structurally reinforced tubing.

FIGS. 6C and 6D show a structural reinforcement for a tubing, wherein the structural reinforcement includes reinforcement of a portion of the tubing that includes side openings.

FIG. 7A illustrates a clippable stylet distal tip portion.

FIG. 7B illustrates a portion of an irrigation catheter having an integrated stylet distal tip portion.

FIG. 7C is a cross-sectional view taken along line 7C-7C in FIG. 7B.

FIG. 7D illustrates a longitudinal sectional view of another clippable stylet distal end portion.

FIG. 7E illustrates longitudinal sectional views of a kit of clippable stylet distal end portions.

FIG. 7F illustrates an interchangeable, fixed stylet distal end portion.

FIGS. 8A-8E show various different radiopaque marker arrangements in irrigation systems.

FIG. 8F is a partial, longitudinal sectional illustration of an irrigation system including an irrigation catheter and removable stylet.

FIG. 9A illustrates an irrigation system including an irrigation catheter and removable stylet.

FIG. 9B illustrates an irrigation system including an irrigation catheter and removable stylet, along with a spacer inserted between hubs of the irrigation catheter and removable stylet.

FIG. 9C is a side view of a spacer.

FIG. 9D is an end view of the spacer of FIG. 9C.

FIG. 9E is a view of a telescoping spacer.

FIG. 9F illustrates a locking arrangement for a telescoping spacer.

FIG. 9G illustrates another adjustable length spacer.

FIG. 9H illustrates a variation of the spacer shown in FIG. 9G.

FIG. 9I illustrates an adjustment mechanism integrated into the hub of the removable stylet.

FIG. 9J illustrates an adjustment mechanism integrated into the hub of the irrigation catheter.

FIG. 10A illustrates a flexibility property of a stylet distal tip portion.

FIG. 10B illustrates a shapeability property of a stylet distal tip portion.

FIGS. 10C-10D illustrate a supportive property of a stylet distal tip portion.

FIG. 11A is a partial, longitudinal sectional view of an irrigation system including a removable stylet and an irrigation catheter.

FIG. 11B is a cross-sectional view taken at line 11B-11B in FIG. 11A.

FIG. 11C is a cross-sectional view taken at line 11C-11C in FIG. 11A.

FIG. 11D is a cross-sectional view taken at line 11D-11D in FIG. 11A.

FIG. 12 illustrates an arrangement for delivering high pressure irrigation to an anatomical site within a patient.

FIGS. 13A-13C illustrate partial view of additional arrangements of irrigation systems in which an irrigation catheter has an integrated stylet distal tip portion.

FIG. 13D illustrates an embodiment where distal tip portion includes a solid polymer tip that contains a radiopaque coil at a distal portion of the distal tip portion.

FIG. 14 illustrates a removable illuminating stylet that includes an illuminating distal tip at a distal end of the distal tip portion.

FIGS. 15A-15D are illustrations of partial sagittal sectional views through a human head showing various steps of a method of gaining access to a paranasal sinus by an irrigation system as described herein to perform at least one of irrigation, suction, delivery of a therapeutic or diagnostic substance or retrieval of a culture.

FIGS. 16A-16E are illustrations of various views of a flexible irrigation catheter according to one embodiment of the present invention.

### DETAILED DESCRIPTION OF THE INVENTION

Before the present devices and methods are described, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are now described. All publications mentioned herein are incorporated herein by reference to disclose and describe the methods and/or materials in connection with which the publications are cited.

It must be noted that as used herein and in the appended claims, the singular forms “a”, “an”, and “the” include plural referents unless the context clearly dictates otherwise. Thus, for example, reference to “a lumen” includes a plurality of such lumens and reference to “the opening” includes reference to one or more openings and equivalents thereof known to those skilled in the art, and so forth.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

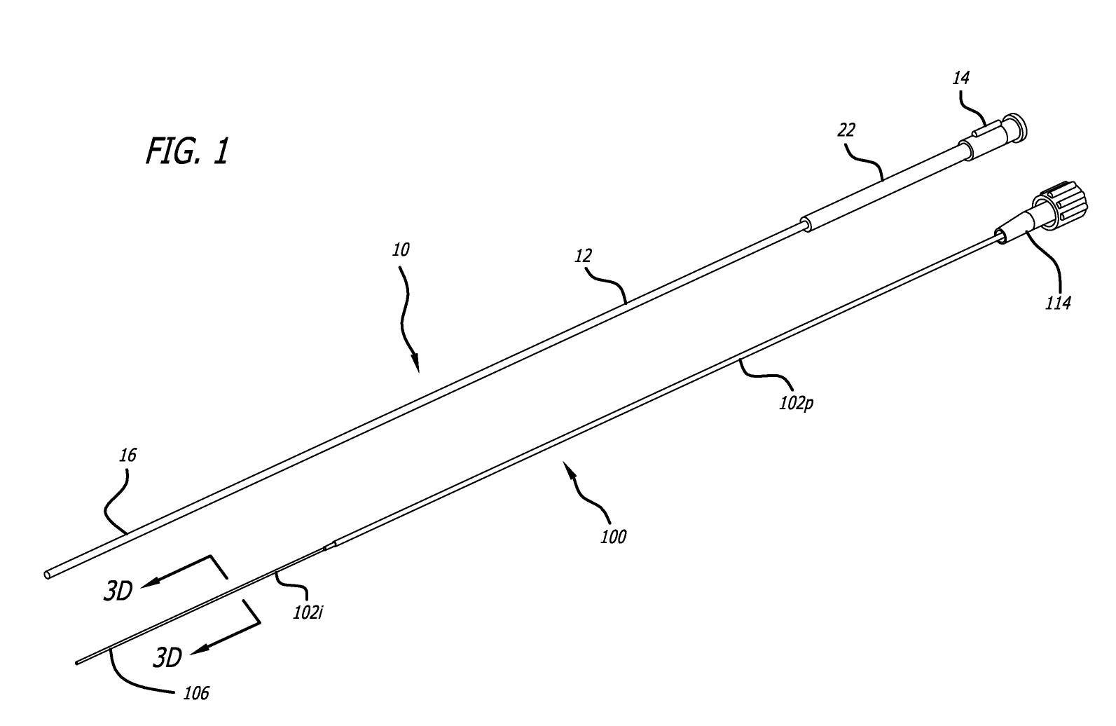

The devices disclosed herein can be used to irrigate and/or suction fluids deep within the sinuses, as well as in other areas within the paranasal space or other locations in the ear, nose and throat anatomy. Devices disclosed herein may also be used to deliver therapeutic substances (e.g., antibiotics, steroids, etc.) to any of the locations mentioned previously, as well as to take cultures from any of those locations. FIG. 1 shows components that are useable together in an irrigation system according to one embodiment of the present application, to perform functions noted above in treatment and/or diagnosis procedures of the ear, nose and throat anatomy.

Irrigation catheter 10 is flexible, so that it can be delivered through the tortuous anatomy, without kinking, for insertion of the distal end portion thereof deep within a sinus cavity or other deep anatomical structure in the ear, nose and throat anatomy. The elongated flexible tube portion is more flexible than currently existing catheters used in the ear, nose and throat space, yet has sufficient wall strength so that the distal end portion can be routed through a guide catheter having a bend up to at least 110 degrees without kinking the tube of the irrigation catheter 10. Guide catheters having such bends in the distal end portion are described, for example, in application Ser. No. 11/193,020, as well as other applications incorporated by reference above. The elongated tube portion of irrigation catheter thus comprises a flexible, biocompatible polymer material, such as nylon, polyethylene, polyether ether ketone (PEEK), or polyether block amides (e.g., Pebax) for example, typically Pebax., as described in more detail below. The elongated tube portion is preferably clear so that a surgeon can see materials being delivered from a target location of the distal end of the irrigation catheter, out through the tubular portion and out of the patient, as well as materials being delivered to the target location, out through the distal end portion of the irrigation catheter. This clear tubing also allows for visual trouble-shooting of the device, e.g., should the tubing become clogged, the user can visualize where along the tubing the clog has occurred, etc.

Irrigation catheter 10 is designed so that it does not have to be delivered over a guidewire. Rather, stylet 100 is provided that is insertable through irrigation catheter 10 and which facilitates the delivery and positioning of the irrigation catheter as described in more detail below. Accordingly, no exchange procedure is required, such as removing a working tool from an appropriately placed guidewire and then “exchanging” by delivering an irrigation catheter over the guidewire to direct it to a target site to perform irrigation and/or suction. For example, in a procedure where a guide catheter is first inserted intranasally and maneuvered to align a distal tip of the guide catheter with a sinus ostium, this can be followed by inserting a guidewire through the guide catheter and into the sinus opening up from the sinus ostium that the guide catheter is aligned with. One or more working tools can then be passed over the guidewire to perform one or more surgical procedures in the sinus or at the sinus ostium. For example, a balloon catheter may be delivered over the guidewire to locate a working end (e.g., expandable balloon) in the sinus ostium. After expansion of the balloon to dilate the sinus ostium, deflation of the balloon and removal of the balloon catheter from over the guidewire, an irrigation catheter could then be exchanged to pass over the guidewire to perform irrigation, suction etc. at the location of the sinus ostium. However, the withdrawal of the balloon catheter off the guidewire to allow for the exchange is not a simple task. For example, the guide catheter will typically need to be held stationary and also the guidewire will need to be held stationary to maintain the desired distal end position, and the guidewire may need to be held at a location other than where the guide catheter is being held. While holding both of these components stationary, the balloon catheter (or other working device) must be pulled on or retracted, to remove it from its location over the guidewire.

By providing a system such as that shown in FIG. 1, the precarious exchange process can be eliminated. Instead, a surgeon can simply pull the balloon catheter (or other working device) and the guidewire out in a single step, or one after the other, but with the point being that no care need be taken to retain the guidewire in position as the working device is withdrawn. After removal of the working device and the guidewire, the irrigation system (irrigation catheter 10 having stylet 100 inserted therein) can be delivered through the guide catheter to perform subsequent functions at the sinus ostium or within the sinus, e.g., irrigation, suction, substance delivery, retrieve a culture, etc.

The system of FIG. 1 also provides advantages for pediatric patients or for adult patients where an ostium dilation is not performed. For example, the irrigation system can be delivered though a guide catheter without first inserting a guidewire and a device to perform an ostium dilation.

Irrigation catheter 10 is configured to irrigate and suction fluids deep within the sinuses, as well as other areas with the paranasal space. Irrigation catheter is sized appropriately to be delivered into adult as well as pediatric sinuses, including maxillary, sphenoid and frontal sinuses. Irrigation catheter 10 can also be used to deliver diagnostic or therapeutic substances into the sinuses or other areas in the paranasal space. Examples of such diagnostic or therapeutic substances include, but are not limited to: contrast agents, pharmaceutically acceptable salt or dosage form of an antimicrobial agent (e.g., antibiotic, antiviral, anti-parasitic, antifungal, etc.), a corticosteroid or other anti-inflammatory (e.g., an NSAID), a decongestant (e.g., vasoconstrictor), a mucous thinning agent (e.g., an expectorant or mucolytic), an anesthetic agent with or without vasoconstrictor (e.g., Xylocaine with or without epinephrine, Tetracaine with or without epinephrine), an analgesic agent, an agent that prevents of modifies an allergic response (e.g., an antihistamine, cytokine inhibitor, leucotriene inhibitor, IgE inhibitor, immunomodulator), an allergen or another substance that causes secretion of mucous by tissues, anti-proliferative agents, hemostatic agents to stop bleeding, cytotoxic agents e.g. alcohol, and biological agents such as protein molecules, stem cells, genes or gene therapy preparations.

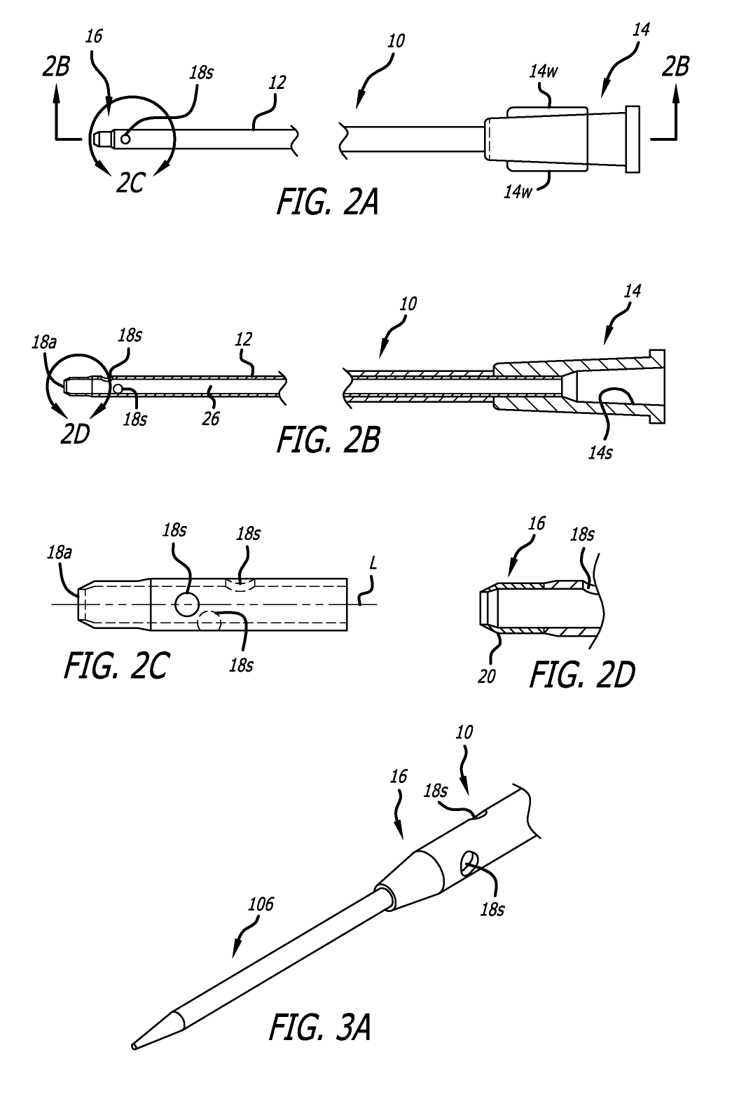

Irrigation catheter 10 includes an elongated flexible tubing that extends from a hub 14 attached at a proximal end thereof to a tapered distal tip 16. One or more openings 18s are provided through a side wall of the tubing 12 at a distal end portion (tip portion) thereof, just proximal of the tapered tip 16, as more easily seen in FIGS. 2A-2D. Additionally, an axially directed opening 18a is provided as an open distal end of device 10. Typically at least two or more, typically three or four openings 18s are provided to direct irrigation spray in different directions radially from tubing 12. More than four openings 18s may also be provided, but three or four of the type openings 18s described herein have been found to optimize a balance for providing spray circumferentially about the longitudinal axis of the tube 12 while maintaining sufficient wall strength of the tubing to prevent kinking, collapsing or other forms of structural failure. Further in this regard, openings 18s can be formed in a spiral pattern about the tubing 12 as illustrated in FIG. 2C, or other pattern, so that no two openings are aligned with one another in a direction perpendicular to the longitudinal axis L. This helps to maintain the wall strength of the tubing 12.

Side openings 18s are provided to create vortices or turbulent flow of irrigation fluid as it is ejected from the side openings. Side openings are placed so as to eject fluid in radially varying directions to produce the turbulent flow vortices in substantially all direction around the circumference of the tubing 10 where side openings 18s are located. For those embodiments that have an end or axial opening 18a, this is also designed to produce turbulent flow/vortices, to act in concert with the vortices produced by side openings 18s. The turbulent flow/vortices are further propagated when the tubing 12 containing the side openings 18s deliver spray in a small anatomical space, such as a sinus cavity, since the spray hits against one or more walls defining the cavity, further disturbing the flow and increasing the turbulence.

Side openings 18s are typically created as circular holes, although other shapes can be formed, including oval openings, slits, other geometrical shapes, teardrop shaped openings, etc. Openings 18s are typically cut or punched through the tubing wall in a direction perpendicular to the longitudinal axis of the tubing 12. However, openings may be cut or punched in an angled direction (other than 90 degrees) to the longitudinal axis of tubing 12. Still further, openings can be cut or punched to have a nozzle-type configuration, where the cross sectional dimension of the opening 18s on the inner wall of tubing 12 is greater or less than the cross-sectional dimension of the opening 18s on the outer wall of tubing 12.

Hub 14 may be provided with a standard luer hub connection that allows a standard syringe to be readily mounted thereto. Hub 14 may be made of polyvinyl chloride (PVC), polycarbonate, stainless steel or other biocompatible metal or other rigid, biocompatible polymer, for example. Hub 14 can be provided with low profile wings 14w that allow manipulation, such as torquing, by a user, but which extend only slightly radially from the main body of hub 14 so that suction hosing can be slid thereover and sealed against the hub to draw suction through the hub.

One or more radiopaque markers 20 may be provided in device 10. For example a radiopaque band is shown in the tip 16 of device 10 in FIG. 2D. Tip 16 is provided with an atraumatic, blunt shape at the distal end thereof, e.g., rounded or otherwise blunted. The proximal end portion of tubing 12 may be overlaid with a stiffer layer or tubing to provide strain relief. For example, FIG. 1 shows strain relief layer 22 that extends distally from a location of hub 14, over a proximal portion of tubing 12. Strain relief layer is formed of a stiffer material than the material forming tubing 12. In the example shown, strain relief layer 22 comprises heatshrink polyolefin tubing. To reinforce the joint between hub 14 and tubing 12.

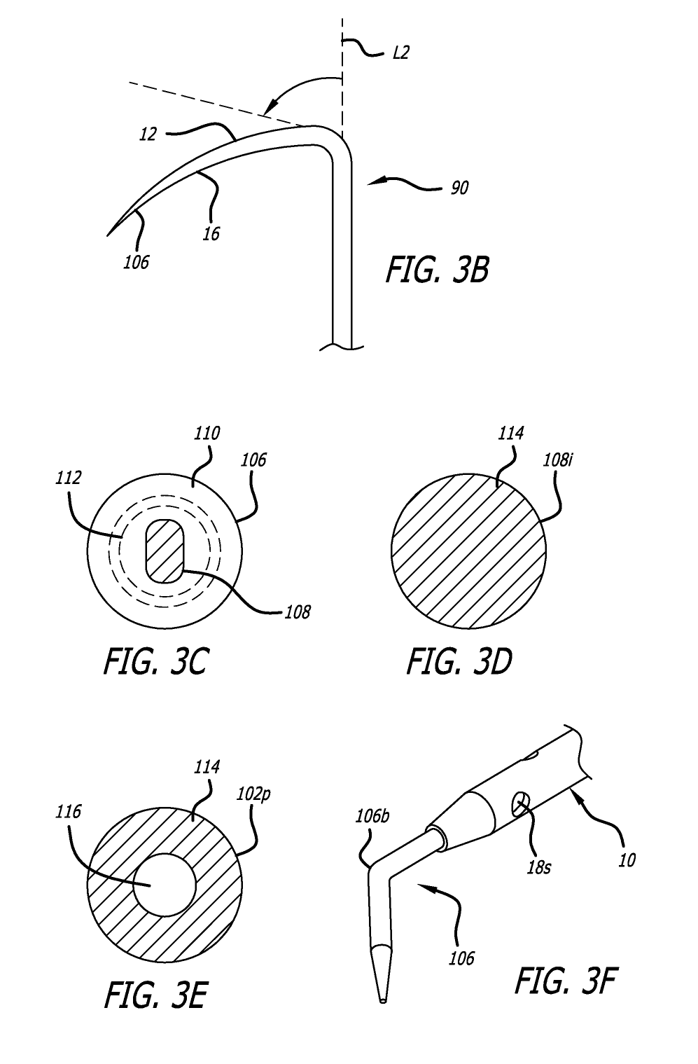

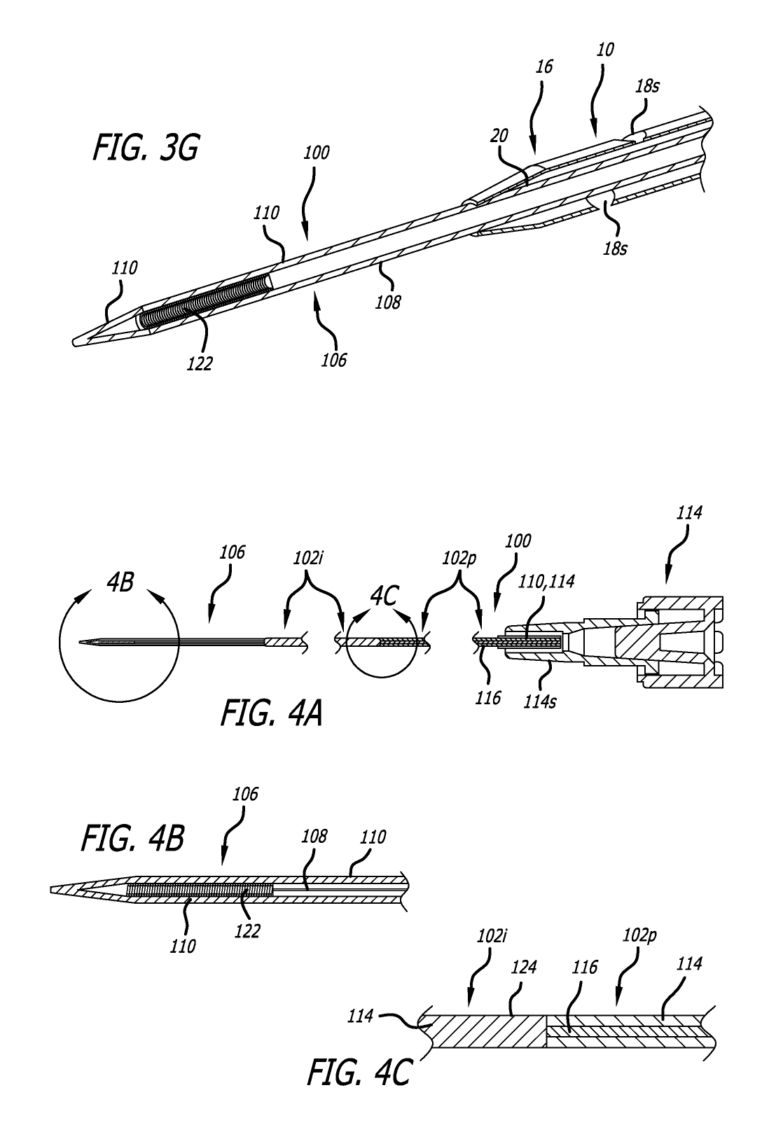

Stylet 100 is configured to be slidably received within irrigation catheter 10 and has a predefined length, so that when connected to or mated with irrigation catheter 10 in a manner described in more detail below, a distal tip portion 106 of stylet 100 extends distally from the tip 16 of irrigation catheter 10. FIG. 3A is a partial view of the irrigation system showing the distal tip 106 of stylet 100 extending distally from the distal end of tip 16 of irrigation catheter 10. The distal end of distal tip 106 extends from the distal end of irrigation catheter 10 by a predetermined length, and this predetermined length can be adjusted by various techniques described herein.

Typically, the flexibility of stylet 100 varies along the length thereof. For example, the proximal portion of the shaft 102 is generally stiffer than the distal portion. In the embodiment shown in FIG. 1, a proximal portion 102p is relatively stiffest with an intermediate portion 102i having an intermediate flexibility, and the tip portion 106 being relatively the most flexible. The proximal portion 102p is stiffest as this is where the operator pushes from when inserting the stylet into the irrigation catheter and when inserting the stylet and irrigation catheter into a patient. Accordingly, it is desirable to have this portion relatively more stiff to provide better column strength so that the stylet does not buckle or bend when pushing on it form the proximal end.

The intermediate portion 102i is somewhat more flexible, as this is the portion of the stylet that may be required to pass through a bend in a guide catheter, when the irrigation system is being delivered though a bent guide catheter, and it is desirable that this portion does not plastically deform when it passes through or is located within a bend in a guide catheter. FIG. 3B illustrates an irrigation system having been inserted though a guide catheter 90. The angle of the bend of guide catheter 30 is measured by the direction that the distal tip extends in, relative to the longitudinal axis L2 of the main portion of guide catheter 90, as illustrated in FIG. 3B.

The distal tip portion 106 is still more flexible than the intermediate portion 102i. Further, the distal tip portion can be made so that it is shapeable, so that it can be plastically deformed with a bend that facilitates steering the stylet 100 as well as the irrigation catheter 10 through the tortuous anatomy, thus providing the same advantages that a guidewire has as it is inserted into the anatomy. However, since the distal end of irrigation catheter is proximally adjacent distal tip 106 as it is advanced, this can provide superior ease of delivery of the irrigation catheter, as compared with delivering an irrigation catheter over a guidewire that has already been previously placed, such that the steerability of the distal tip of the guidewire is not available as the irrigation catheter is inserted over the guidewire. For this reason, an irrigation catheter advanced over a guidewire can tend to get caught up, or snag on various formations in the tortuous anatomy as it is advanced over a guidewire, particularly where the tolerances between the inside diameter of the catheter and the outside diameter of the guidewire are relatively large.

A settable tip can be provided by the inclusion of a shaper wire 108 within the proximal portion. For example shaper wire may be a flattened wire made of stainless steel, nickel-titanium alloy, or other biocompatible metal having characteristics allowing the wire to be plastically deformed when bent over by hand by a user. FIG. 3C illustrates a cross-sectional view of distal end portion 106 in a location where shaper wire 108 extends, taken along section line 3C-3C in FIG. 1. Shaper wire 108 is encapsulated in the polymer 110 that forms the outer portion of the shaft of tip 106. Polymer 110 in the distal tip may be softer than the polymer used to form the remainder of the stylet to make it more flexible and/or distal tip 106 may be somewhat smaller in outside diameter and taper gradually to the outside diameter of the intermediate portion 102i.

In the embodiment shown in FIG. 1, intermediate portion 12i is formed of flexible polymer beading with no internal or core component. FIG. 3D illustrates a cross section of the intermediate portion 12i taken along section line 3D-3D in FIG. 1. The proximal portion 102p in the embodiment of FIG. 1 is made of the same polymer 114 as that of the intermediate portion 102i, which is somewhat harder than polymer 110 thereby providing more stiffness. Proximal portion 102p is made stiffer than intermediate portion 102i by the use of a core wire 116 that has greater stiffness than polymer 114. Core wire 116 has a substantially larger gauge than shaper wire 108 so that it is not readily plastically deformed during use, but provides additional stiffness and column strength to proximal portion 102p. FIG. 3E illustrates a cross-sectional view of proximal portion 102p taken along section line 3E-3E in FIG. 1. Although the same hardness polymer 114 may be used, by encapsulating core wire 116 (which may be stainless steel, nickel-titanium alloy, or other biocompatible metal having the requisite stiffness properties), this increases the overall stiffness of proximal portion 102p relative to the stiffness of intermediate section 102i. Additionally or alternatively, the polymer used in making the proximal portion 102p may have a greater hardness than the polymer used for intermediate portion 102i. Further alternatively or additionally, the intermediate portion 102i may include a core wire having a much smaller cross sectional dimension than core wire 116 in proximal portion 102p, as this can be used to more easily target the desired stiffness characteristics of the intermediate portion 102i, while still making it more flexible than proximal portion 102p.

FIG. 3F illustrates an example of distal tip portion 106 having been bent to set a bend 106b to facilitate steering the stylet 100 and irrigation catheter 10 during insertion of the irrigation system into a patient. By rotating the stylet having the bend 106b, the direction in which the distal end of the stylet points can be varied, thereby facilitating the direction in which the assembly is advanced as it is pushed against the internal anatomy. This is referred to as “steering” the stylet as it is advanced.

FIG. 3G shows a longitudinal sectional view of a distal end portion of stylet 100 having been inserted through irrigation catheter 10. FIG. 3G shows that shaper wire 108 does not extend to the distal end of distal end portion 106 as the distal tip is configured to retain flexibility and to not plastically deform as it is bent over during use. The distal tip may include one or more radiopaque markers 122. When located in the flexible portion distal of shaper wire 108, radiopaque marker 122 can be provided in the form of a coil as shown in FIG. 3G. This coil configuration maintains the flexibility of the distal tip, so that it does not plastically deform during use, and provides superior flexibility relative to that provided by a marker band. The radiopaque marker may be made of platinum, tungsten, iridium, palladium, silver, stainless steel, nickel, titanium, alloys thereof, or other dense, biocompatible material having similar physical characteristics and which is readily identifiable under x-ray or fluoroscopic visualization. These materials may also be coated with or otherwise include barium sulfate or other radiopaque compounds typically used in the art.

FIG. 4A is a longitudinal sectional view of portions of stylet 100, showing the core wire 116 running the length of proximal portion 102p, intermediate portion 102i made of polymer beading 102i without core wire support, and the distal end portion including shaper wire 108 extending through a proximal portion thereof (better seen in the enlarged view of FIG. 4B) and radiopaque marker coil 122 distal of shaper wire 108. Hub 114 includes a male slip luer connector 114s configured and dimensioned to be received in female slip luer connector 14s on irrigation catheter 14. Thus, stylet 100 can be rapidly and securely connected to irrigation catheter by inserting stylet 100 through irrigation catheter 10 until male slip connector 114s and female slip connector 14s form a mating, friction fit. The length of stylet 100 is configured so that a predetermined length of distal end portion 106 extends from the distal end of irrigation catheter 10 when the friction fit is established. The slip fit luer connectors do not require any torquing of the stylet 100 relative to the irrigation catheter 10 to establish the connection, and thereby provide further assurance that the stylet does not kink, twist, or experience any other undesirable deformation during the connection process. However, the luer connectors 14, 114 may alternatively be fitted with mating threads, bayonet connection mechanism, ball-detent connectors, or other alternative mechanical connecting mechanisms, if desired. Hub 114 may be made from any of the materials described above with regard to hub 14.

FIG. 4B shows an enlarged view of the distal end portion identified by circle 4B in FIG. 4A. Shaper wire 108 is located proximally of radiopaque marker 122 and both are surrounded by the polymer material 110 forming the exterior of the shaft of distal end portion 106.

FIG. 4C shows an enlarged view of the portion of stylet 100 wherein the intermediate portion 102i joins the proximal portion 102p as identified within circle 4C in FIG. 4A. A joint reinforcer 124 may be provided over the location where intermediate portion 102i joins proximal portion 102p which extends proximally and distally over portions of intermediate portion 102i and proximal portion 102p extending from the joint. The joint can be melted together and/or joined with adhesive. Joint reinforcer 124 may be in the form of heat shrink tubing, for example.

In one particular embodiment, the polymer 110 in distal tip portion is Pebax, 55 Durometer hardness and the polymer 114 in intermediate 102i and distal 102p portions is Pebax, 72 Durometer hardness. The joint between proximal portion 102p and hub 114 may contain an additional layer of polymer, which in this particular embodiment is Pebax, 55 Durometer. However, this layer could also be made of Pebax, 72 Durometer or some other polymer. It should also be noted that the present invention is in no way limited to these specifications of one particular embodiment, as any or all of these specifications may vary in other embodiments. In this particular embodiment, the overall length of irrigation catheter is about 34.5 cm. This length may vary from about 20 cm to about 60 cm or about 30 cm to about 75 cm or about 35 cm up to about 80 cm or about 25 cm to about 45 cm. The opening 18a is about 0.038 inches in diameter, although this size may vary from about 0.016 inches to about 0.042 inches. Irrigation catheter 10 in this particular embodiment has three side openings or holes 18s, helically spaced about 1 mm apart, and each having a diameter of about 0.040 inches, although diameters may range from about 0.025 inches to about 0.045 inches or about 0.040 inches to about 0.050 inches or about 0.045 inches to about 0.060 inches, and openings 18s can be arranged in some pattern other than a helical one. In this particular embodiment, the radiopaque marker 20 is located about 0.6 mm from the distal end of stylet 10. Of course this distance may vary, but is a predetermined distance from the distal end of stylet 10, so that a user can visualize the marker and know approximately where the distal end of stylet 10 resides. The inside diameter of irrigation catheter 10 in this particular embodiment is about 0.054″, although diameters may vary, as noted in other examples herein. The outside diameter of irrigation catheter 10 in this particular embodiment is about 0.078″, although diameters may vary in other embodiments.

The length of the stylet 100 is greater than the length of irrigation catheter 10, and is configured so that, when stylet 100 is mated with irrigation catheter 10 in a manner as described above, the distal end of stylet 100 extends beyond the distal end of irrigation catheter 10 by a predetermined distance. In this particular embodiment currently being described, the predetermined distance is about 18 mm. However, this predetermined distance may vary in other embodiments. In this particular embodiment, shaper wire 108 is about 0.004 inches in thickness and about 0.013″ in width and is made of stainless steel although these dimensions and material may vary in other embodiments.

The distal end of radiopaque marker coil in this particular embodiment is about 2.5 mm from the distal end of stylet 100 and is formed of a platinum-tungsten alloy, although this dimension and material may vary in other embodiments. The outside diameter of the main shaft 102 is about 0.039″, although outside diameters may vary in other embodiments, and will vary according to the inside diameter of the irrigation catheter 10 that it is designed to be inserted into. The core wire 116 in proximal portion 102p is about 0.012″ in diameter, although this diameter may vary in other embodiments. In this embodiment, intermediate portion 102i is flexible beading of Pebax, 72 Durometer hardness and has a length of about 10 cm, although this length and material may vary in other embodiments.

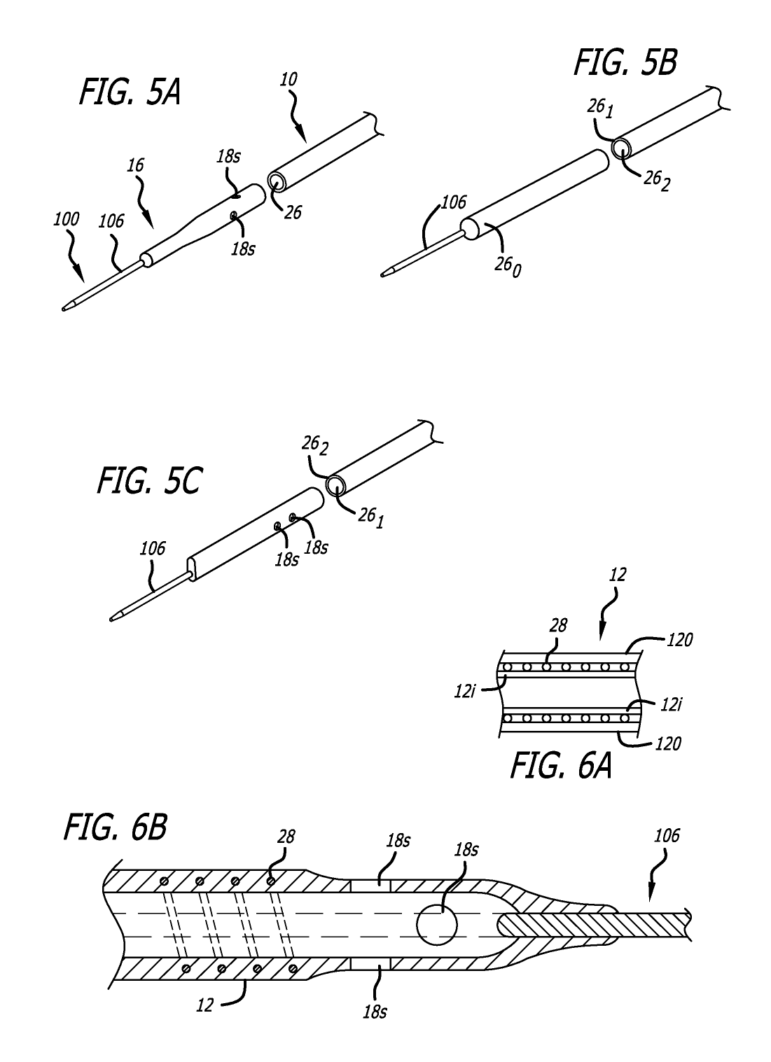

FIGS. 5A through 5C illustrate embodiments of an irrigation system in which only a distal portion of stylet 100 is provided to extend from the distal tip 16 of irrigation catheter 10. In these embodiments, stylet portion 106 is fixed in the distal opening 18a of irrigation catheter 10 and is not removable therefrom to allow irrigation or suction though opening 18a as opening 18a remains plugged by stylet portion 106 during use. Accordingly, irrigation, suction and other functions such as substance delivery, for example, are performed only though side openings 18s. Further, since stylet portion 106 is not removable, these embodiments do not allow irrigation catheter 10 to be alternatively used over a guidewire. Stylet portion 106 is shapeable for providing steerability to the irrigation system, and may be made shapeable by any of the same techniques described above with regard to the distal portion 106 of the removable stylet 100.

Stylet portion may be made of stainless steel, nickel-titanium alloy or other biocompatible metal or polymer that is flexible, but configured to be plastically deformed, so that portion 106 can function in steering the irrigation system through the tortuous anatomy. Stylet portion may extend from the distal end of irrigation catheter by a length of about one cm to about six cm, for example. An extension length of about four cm may be suitable for accessing a frontal sinus of a patient, an extension length of about one to two cm may be suitable for accessing pediatric maxillary sinuses. Distal stylet portion 106 can also be provided with a radiopaque marker 122 that can provide a fluoroscopic visualization to indicate the location of the side holes 18s of irrigation catheter 10, as the user will know the predetermined distance between such marker and the side openings 18s. Other placements of radiopaque markers can be made for this function as well, as described in more detail below. In the embodiment shown in FIG. 5A, a single lumen 26 is provided in irrigation catheter through which both irrigation and suction can be performed, as well as other functions including, but not limited to delivery of diagnostic or therapeutic substance, and taking cultures. FIGS. 5B-5C illustrate an embodiment in which dedicated lumens 26₁ and 26₂ are provided for performing suction and irrigation simultaneously. FIG. 5B illustrates one side of a distal portion of the irrigation system showing an end opening 26o in fluid communication with lumen 26₂ and FIG. 5C shows the opposite side of the distal portion of the irrigation system of FIG. 5B showing side openings 18s in fluid communication with lumen 26₁.

The side openings 18s can be varied in diameter, number and arrangements as already noted. Relatively small numbers, e.g., about one to ten of relatively larger diameter holes are preferred over large numbers, e.g., greater than twenty, greater than fifty or greater than one hundred holes having relatively smaller diameters, as both sets of these arrangements were found to be effective for irrigation, but the arrangements with smaller numbers of larger holes provided an advantage for suction, since large particles of debris can be taken up through the large holes. However, the arrangements having larger numbers of smaller diameter holes are not excluded from this disclosure. In another embodiment, an arrangement of four side holes 18s each having about 0.050 inch diameter and being equally distributed around the circumference of the irrigation catheter tip 16 (at a location where catheter tip is not tapering down, but has the full diameter of the remainder of the shaft 102). When irrigation catheter 10 is connected to a syringe (having a volume of about 10 cc to about 60 cc, for example), an irrigation stream can be delivered that can vary in pressure from a gentle rinse to a vigorous wash, depending upon the amount of pressure applied to the plunger of the syringe by the user.

In one embodiment, distal stylet portion 106 is made of a coil made of a core wire of stainless steel of about 0.025 inch diameter and irrigation catheter is made from Pebax, 55 Durometer hardness. A polyimide strain relief tube 22 of the type described above with regard to FIG. 1 is provided that has about 0.0015″ wall thickness. A luer hub 14 is mounted to the proximal end of irrigation catheter 10 in the same manner as described above.

One of the design challenges for the irrigation catheter 10 is to provide the irrigation catheter 10 to be guided around a bend in a guide catheter, wherein the bend is up to at least about 110 degrees, measured as described above, without kinking occurring in the tubing 12 of the irrigation catheter 10. One way to address this concern is to increase the wall thickness of the polymer material forming the tubing 12 so that is strong enough not to kink. Another approach is to reinforce the polymeric wall of the tubing 12, such as by including a coil, braided tubing, spiral cut tubing, or other reinforcing structure 28 within (between) the inner and outer wall surfaces of the tubing 12. An advantage to using a reinforcing structure 28 is that it enables the overall wall thickness of tubing 20 to be made thinner than one made solely of polymer, in order to attain the same strength/kink resistance. This is advantageous since the outside diameter of tubing 12 is constrained to a limit to enable it to be passed through a guide catheter, for example, while it is also of interest to maintain the inside diameter of tubing 12 as large as possible to maximize the ability to transport fluids therethrough. The resistance to flow within a tube is proportional to the length of the tube and inversely proportional to the fourth power of the inside diameter of the tubing:

$$Resistance ∝ \frac{Length}{{ID}^{4}} .$$

Accordingly, since the outside diameter of the design is constrained, it becomes very important to minimize the wall thickness, as small increases in the inside diameter can have a great reducing effect on the resistance. In two specific embodiments, a tubing 12 made of Pebax and having a wall thickness of about 0.012″ was sufficient to prevent kinking, for an outside diameter of about 0.078″, when the tubing was passed through a guide catheter having a 110 degree bend. A coil reinforced Pebax tubing 12 having the same diameter had a wall thickness of about 0.008″ and prevented kinking when being passed through the guide catheter having the 110 degree bend.

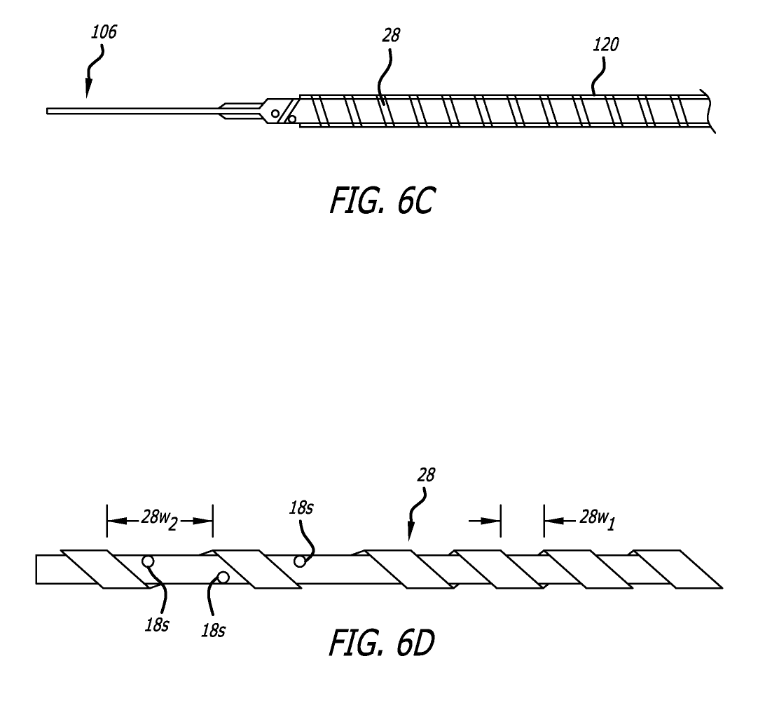

FIG. 6A illustrates one configuration for providing a reinforced tubing wherein reinforcing structure (a coil in the example shown in FIG. 6A) is sandwiched between two layers of polymeric material, and inner tube 12i and an outer tube 12o. Alternatively, reinforcing structure 28 can be molded within the polymeric wall of tube 12, as illustrated in FIG. 6B. In the example shown in FIG. 6B, polymer tubing is molded with reinforcing structure 28 therein to encapsulate the same between inner and outer wall surfaces of a single tubular structure 12. The reinforcing structure is present only in a portion of tube 12 proximal of side openings 18s, with the section containing the openings 18s consequently being radiolucent. However, by visualizing the coil, where the coil is a radiopaque metal, an operator can locate the side openings, knowing that they are placed just distal of the distal end of the reinforcing structure 28.

Also, in this embodiment, the distal tip, stylet-like portion 106 is metal wire and also radiopaque. The combination of reinforcing structure 28 and distal tip portion 106 outlines the radiolucent section that corresponds to the openings 18s. Thus, under fluoroscopic, or other x-ray visualization, the openings 18s can be located by a “negative” type of visualization, i.e., the gap that shows up between the visualization of the support structure 28 and distal tip 106.

FIG. 6C illustrates an embodiment where a flat coil or spiral ribbon 28 structurally reinforces tubing 12, including the portion of tubing 12 in which side openings 18s are formed. Reinforcing structure 28 is sandwiched between two layers of polymer 12o and 12i in FIG. 6C, but a similar arrangement can be made by encapsulating reinforcing structure 28 in a single polymeric tubing 12. In order to provide adequate spacing for forming side holes 18s between windings or coils of reinforcing structure 28, the windings or coils of reinforcing structure can be pulled apart to increase the pitch/distance between coils/windings 28w2 in the locations where side opening 18s are to be formed, relative to the pitch/distance 28w1 between coils/windings in the remainder of reinforcing structure 28. FIG. 6D illustrates coils at a distal end portion of reinforcing structure having been pulled apart to increase the distance between windings, and locations where the side openings are to be formed are indicated by the circles 18s. Side holes 18s may be punched through tubing 12, such as by laser drilling, or other known techniques. In one particular embodiment, the layers 12o and 12i of tubing 12 comprise 55 durometer Pebax and reinforcing structure 28 is a stainless steel coiled ribbon of thickness about 0.002″ and width about 0.012″ and the pitch 28w1 is about 0.008″. Pitch 28w2 is an expanded width permitted a side hole 18s having a diameter or about 0.040″ to be punched in tubing 12. Tip 106 is 304V stainless steel having a length of about four cm. The inside diameter of tubing 12 is about 0.081″ and the outside diameter of tubing 12 is about 0.096″.

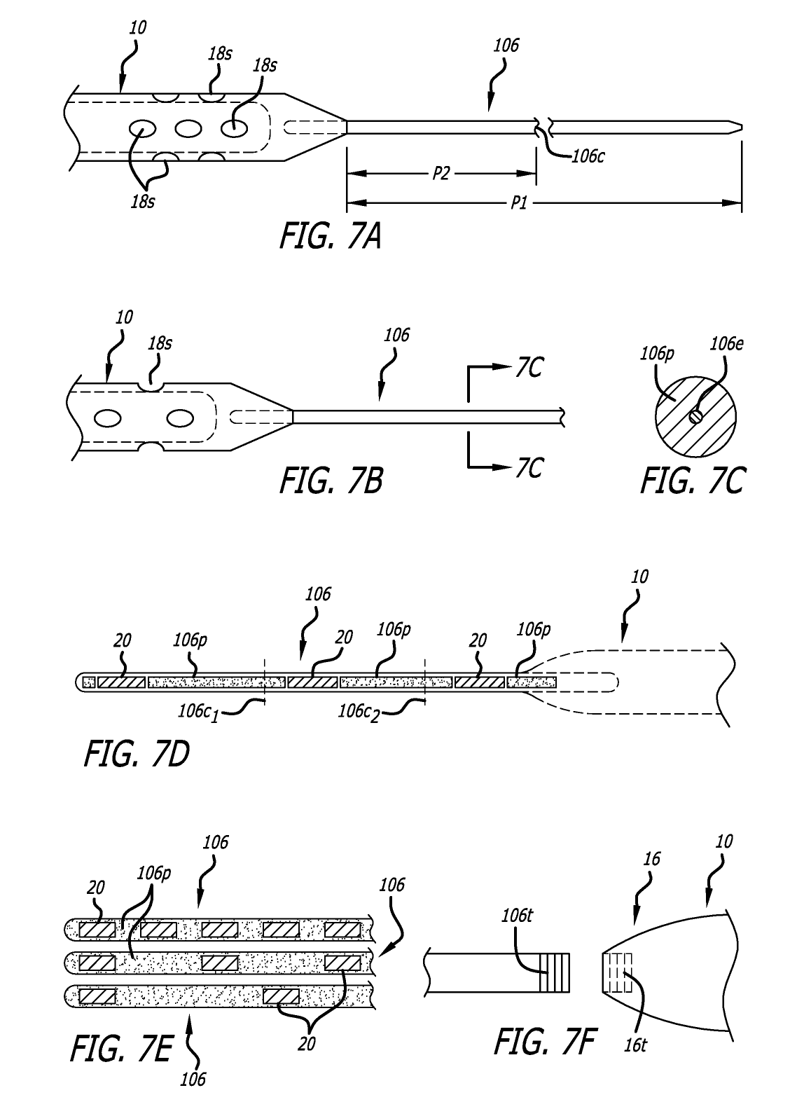

Alternative to the use of a wire tip 106, a polymeric tip can be used. One advantage of using a polymeric tip, is that it can be trimmed by a surgeon to customize the length that the distal tip, stylet-like portion 106 extends from the distal end of catheter 10. Whereas use of the wire tip 106 provides only a single predetermined extension distance, use of a polymeric tip 106 allows the extension distance to be customized by the surgeon, by clipping a portion of the tip 106 to change the length thereof, and thus change the distance by which the distal end of tip 106 extends from the distal end of catheter 10. FIG. 7A illustrates one example wherein a polymer tip 106 extends originally by a predetermined distance of P1, and wherein tip 106 has been clipped at 106c to customize the extension length to P2. Examples of polymers that can be used to make stylet-like tip 106 include, but are not limited to: Pebax, polyurethane, and Nylon. A material should be chosen so that the trimmed tip does not have sharp, traumatic edges, but maintains an atraumatic contour, similar to the distal end of the untrimmed tip 106.

Further alternatively, tip 106 may be formed by a thin metal wire core 106e covered by polymeric material 106p as illustrated in the enlarged cross-sectional view in FIG. 7C taken along section line 7C-7C of the irrigation system partially illustrated in FIG. 7B.

FIG. 7D illustrates an irrigation system having a clippable stylet, distal end tip portion 106 extending distally from a distal end of irrigation catheter 10. In this embodiment, radiopaque markers 20 are intermittently placed between polymeric sections 106p of tip 106 so that radiopaque markers 20 and polymeric sections 106p alternate in an axial direction (distal to proximal or proximal to distal) along the tip 106. Accordingly, when a user clips a portion of tip 106 to adjust the length thereof, at least one radiopaque marker typically remains in the clipped tip portion 106 that remains connected to the irrigation catheter 10. Phantom lines 106c1 and 106c2 indicate two exemplary locations where tip 106 can be clipped and where at least one radiopaque marker 20 remains in place on the attached, clipped tip portion 106. Of course these are only two examples, as tip 106 can be clipped at any locations along the polymeric sections 106p. If the user accidentally clips at the location of a radiopaque marker and the clipping operation is not successful, the user can make another clip just proximal of that radiopaque marker. To increase the adjustability or ability to customize the length of tip 106, a kit of irrigation systems may be provided wherein irrigation catheter 10 is essentially the same in each system, but wherein the pattern of radiopaque markers 20 and polymeric section 106p varies among the different systems in the kit. FIG. 7E illustrates three different tip arrangements 106 as non limiting examples of those that can be included in a kit. Tips may be integrally formed each on its own irrigation catheter. Alternatively, tips 106 may be provided with threads 106t at the proximal end of each that are mateable with threads 16t on the inside surface of the distal end opening in irrigation catheter 10, as illustrated in FIG. 7F. In this case, a tip 106 can be removed from irrigation catheter 10 by unscrewing it, and a tip 106 having another arrangement of radiopaque markers 20 and polymeric sections 106p can be installed in its place by screwing it into the distal end opening of irrigation catheter 10.

Radiopaque markers 20 may be stainless steel, tungsten, or other metal or dense material that is readily visible under fluoroscopy. Polymer sections may be made of any of the various polymers described previously for making tip 106. In one particular embodiment, radiopaque markers are 0.020″ in diameter and an inner mandrel of polymer forming polymeric sections 106p is Barium-loaded Pebax, with the Pebax having a hardness of from about 35 to about 50 durometer, Shore hardness. Markers 20 may have a length of about two to about six mm, typically about three to about 5 mm, and in two particular embodiments, had lengths of 3 mm and 6 mm respectively. Both the radiopaque markers 20 and the polymeric inner mandrel sections 10p can then be coated by an external layer or outer jacket of polymer to form a smooth integral tip 106. In one particular embodiment, the outer jacket is Barium-loaded Pebax, with the Pebax having a hardness of from about 35 to about 50 durometer, Shore hardness.

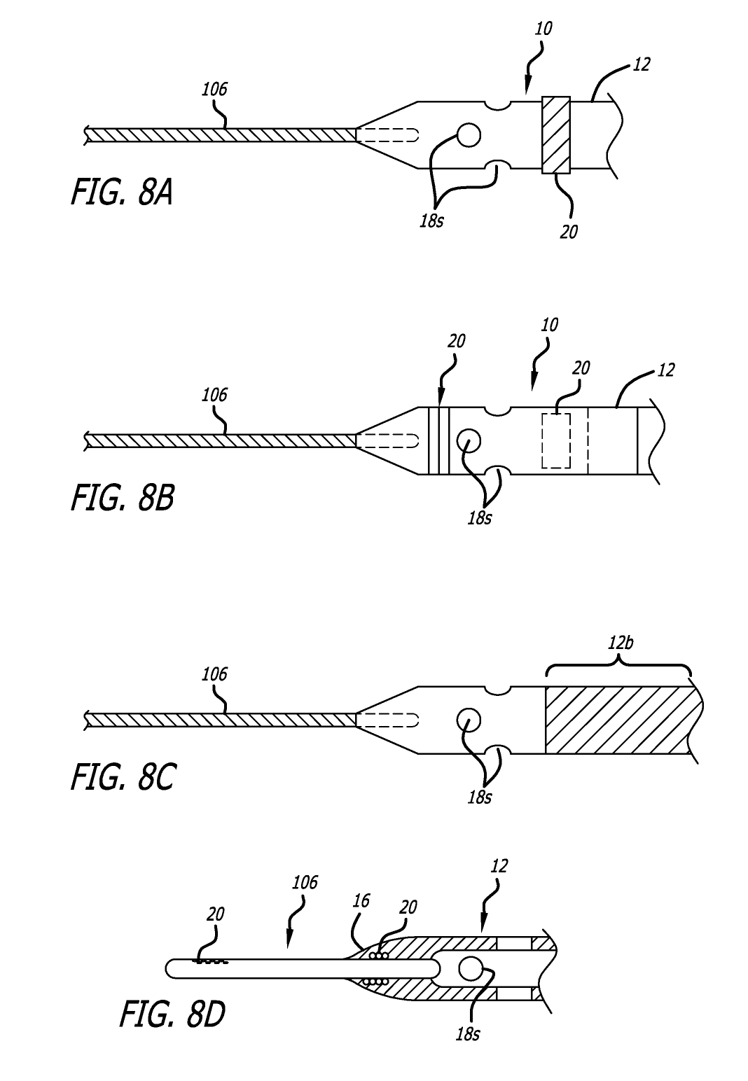

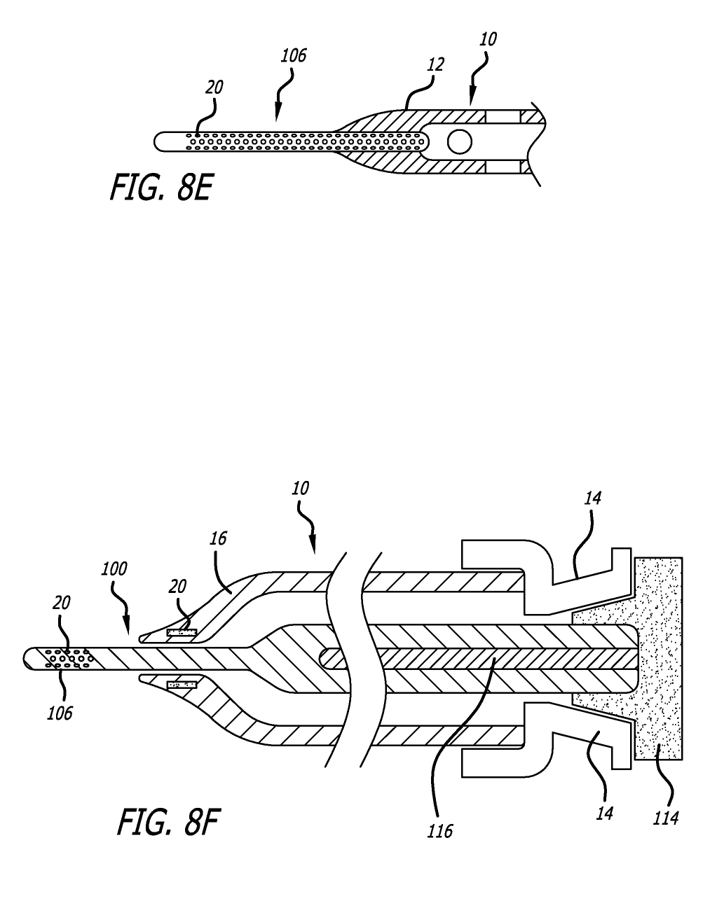

Various marking configurations can be used to facilitate fluoroscopic visualization of the location of the side openings 18s in irrigation catheter 10. For example, a radiopaque marker band (e.g., platinum or iridium band, or the like) can be located on or within the tubing wall 12 proximally adjacent the proximal most side opening 18s as illustrated in FIG. 8A. Additionally, in this embodiment, stylet tip portion 106 is metallic (e.g., stainless steel or the like) and functions as a radiopaque marker. In an embodiment of the type employing a removable stylet 100, such as like that shown in FIG. 1, a marker 20 can be located the same as shown in FIG. 8A, and another radiopaque marker 20 can be located distally adjacent the distal most side opening 18s, for example, in the manner shown and described above with regard to FIG. 2D. A similar arrangement can be provided in a system of the type shown in FIG. 8A, as illustrated in FIG. 8B, wherein dual radiopaque markers 20 are sandwiched in between layers of Pebax forming the tubing 12, at locations proximally adjacent the most proximally located side opening 18s and distally adjacent the most distally located side opening 18s, respectively.

FIG. 8C illustrates another marking configuration, wherein tip 106 is metallic and therefore substantially radiopaque, and a portion 12b of tube 12 that is proximal of the most proximally located side opening 18s is barium-loaded, to provide radiopacity. For example, this portion 12b of tubing can be formed by extruding barium-loaded polymer. The entire length of tubing proximal of the side openings 18s can be barium-loaded, or only a band of tubing just proximal of the side openings 18s can be barium-loaded, with the remaining portion of tubing proximal of the barium loaded section 12b being made of radiolucent polymer. Alternatively, the portion of tube 12 containing side holes 18s may be barium loaded, in which case the portion 12b shown in FIG. 8C would then be barium free and radiolucent. In this alternative arrangement, tip 106 can also be made of radiolucent polymer. However, even with a metallic wire tip 106, a barium loaded section of tubing 12 containing the side holes 18s may still be distinguishable from tip 106 because of its substantially greater width in the visualization.

FIG. 8D illustrates another configuration in which a platinum coil 20 is located within the distal tip of irrigation catheter 10 around a proximal end of stylet tip portion 106. Additionally, a radiopaque marker 20 is located near a distal end of tip 106. This marker may be a platinum, stainless steel or tungsten coil, for example, or may be a polymer segment that is barium-loaded, for example.

FIG. 8E shows another example in which a radiopaque marker is extends along substantially the entire length of tip 106, Marker 20 may be a coil of the type described above with regard to FIG. 8D, or may be a stylet shaft 106b that is formed entirely of barium-loaded polymer, such as barium-loaded Pebax for example. The arrangements described herein are merely for exemplary purposes and are not meant to be exhaustive of all marking configurations that could be used. Further, features of the various embodiments described above may be combined with features of other marking configuration embodiments where possible.

FIG. 8F illustrates an irrigation system having a removable stylet, wherein a radiopaque marker band is embedded in distal end portion 16 of irrigation catheter 10 and a radiopaque metallic coil 20. (e.g., platinum marker coil) is embedded in the distal end portion 106 of removable stylet 100. Note that the tolerance between the outside diameter of stylet 100 and the inside diameter of irrigation catheter 10 is not shown to scale for purposes of more clearly showing the components. In one particular embodiment, when the slip luer features of hubs 14 and 114 are mated as shown in FIG. 8F, the distal tip of stylet 100 extends from the distal tip of irrigation catheter by a distance P1 of about 1.5 cm. As noted earlier, the desired predefined distance may vary, depending upon the particular anatomy that is to be accessed (e.g., frontal sinus versus maxillary sinus) the patient type (e.g., male vs. female, adult vs. pediatric) or even the particular patient. Accordingly a series or kit of stylets 100 may be provided having varying lengths so that, when installed in irrigation catheter 10, the predetermined distance P1 varies. These stylets 100 can be color coded to differentiate between the different lengths, e.g., by colored bands provided on the stylets.

Alternatively, the predetermined distance P1 may be made adjustable by the provision of a stylet 100 that can be adjusted, relative to irrigation catheter 10, so that stylet 100 and irrigation catheter can be mated so that the distal end of stylet 100 extends beyond a distal end of irrigation catheter by a predetermined distance that can be selected by the user, within a range of predetermined distances that the system is adjustable to achieve. In one embodiment, one or more spacers 1114 are provided that interconnect between the mating components of irrigation catheter 10 and stylet 100 to reduce the predetermined length by which the stylet tip extends from the irrigation catheter tip. For example, when the mating components are male and female slip luer connections as described above, spacer 1114 can be formed as a stackable luer hub having a male slip taper 1114m for mating with the female luer taper of the mating components of the system (on connector 14 of irrigation catheter 10, in the examples shown, although the female taper could alternatively be on connector 114 of stylet 100), and a female slip taper 1114f for mating with the male luer taper of the mating components of the system.

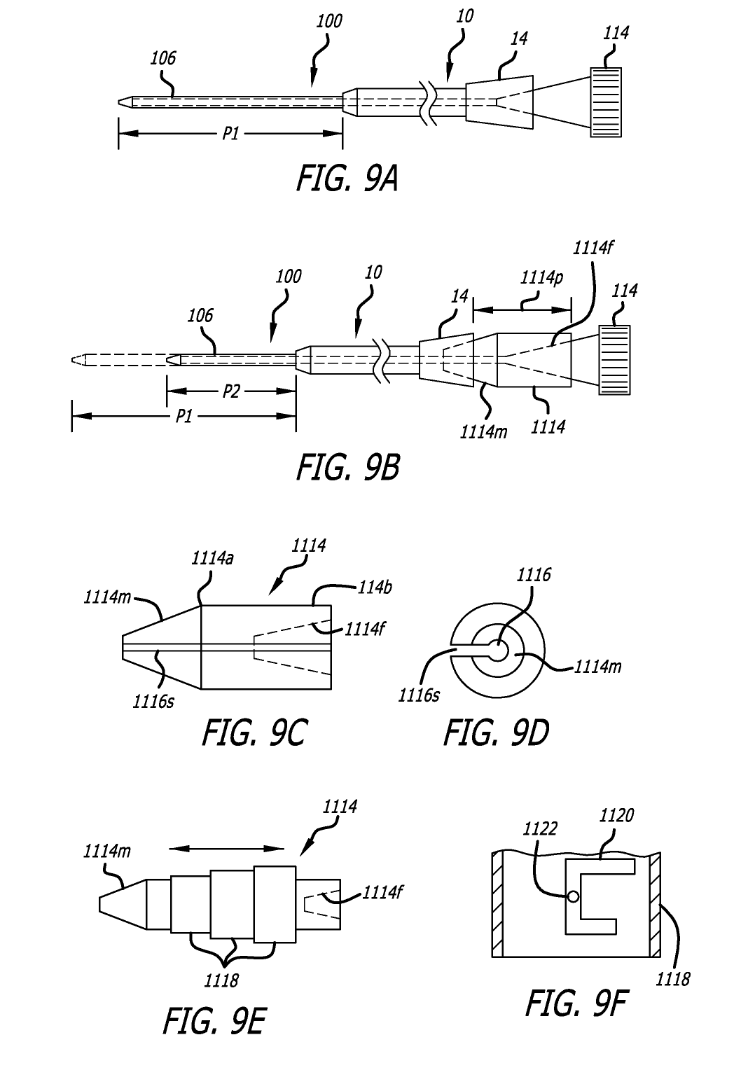

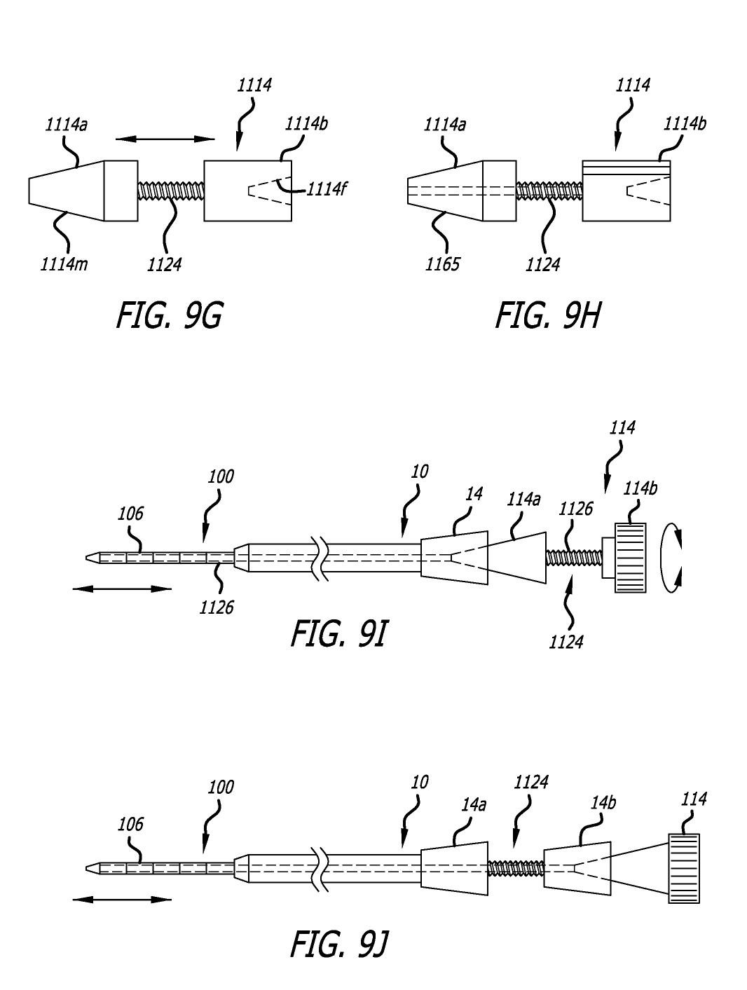

FIG. 9A illustrates stylet 100 mated with irrigation catheter 10 via luer slip mating such that the distal end of stylet 100 extends beyond the distal end of irrigation catheter 10 by a predetermined distance P1. FIG. 9B illustrates a spacer having been inserted between the mating surfaces of hubs 14 and 114, so that male luer taper 114m mates with the female luer taper of connector 14 and female luer taper 1114f mates with the male luer taper of connector 114. When all components are securely mated, the predetermined distance P2 by which the distal end of stylet 100 extends from the distal end of irrigation catheter 10 is less than predetermined distance P1 by an amount equal to the distance 1114p by which spacer 1114 separates the hubs 14 and 114 from their relative positions when directly mated without the use of a spacer 1114. That is, P1−1114p=P2. Spacers 1114 of varying length can be provided in a kit to allow a user to select a particular length 1114p by which to shorten the predetermined length P1. Additionally, spacers 1114 can be labeled with pre-calculated, predetermined lengths that result from their use with a particular irrigation system, so that the user does not have to calculate P1−1114p=P2 for each spacer, but can just select from the pre-calculated P2 values that are labeled on the spacers 1114. Additionally, spacers 1114 may be color-coded with different colors relative to one another, for easier identification and selection of a particular spacer to use. Still further, spacers 1114 are stackable, so that more than one spacer 1114 can be mated in between the hubs 114 and 14. In such a use, the reduction in the predetermined distance is equal to the sum of the individual distances by which each of the spacers 1114 used would reduce the length of the predetermined distance when used alone. In one example, spacers are provided having reduction lengths 1114p in five mm intervals, e.g., 1114p1=5 mm, 1114p2=10 mm, 1114p3=15 mm, etc, and are color-coded by length. Of course, kits of spacers 1114 may be provided in other series of varying lengths, and the incremental changes in lengths between spacers need not all be the same.

Spacers 1114 have a lumen 1116 that extend therethrough to allow the spacer 1114 to be slid over the shaft of the stylet 100 prior to insertion of the stylet 100 into irrigation catheter 10. Alternatively, spacer 1114 may be provided with a slit 1116s that extends through the wall of the spacer and extends the length of the spacer to provide an access opening to lumen 1116, as illustrated in FIG. 9C and the end view of FIG. 9D. With this configuration, stylet 100 can already be inserted into irrigation catheter when spacer 1114 is inserted. All that is required is that hub 114 be space proximally from hub 14 to expose a portion of stylet shaft 102 of sufficient length to allow spacer to be slid thereover, so that shaft 102 passes though slit 1116s and into lumen 1116. This is advantageous, particularly where it becomes desirable to exchange one spacer 1114 for another, or to add a spacer 1114, as it does not require complete removal of stylet 100 from irrigation catheter 10 and then reinsertion of stylet 100 into irrigation catheter each time one of these function is performed.

FIG. 9E illustrates another embodiment of a spacer that has an adjustable length 1114p. The adjustable length is achieved by one or more telescoping components 1118 that are slidable (telescoping) with regard to a fixed portion of the spacer 114. For example, the telescoping portions 1118 in FIG. 9E are axially slidable with respect to the fixed distal end portion that includes the male luer taper 1114m. Telescoping portions 1118 can be individually slid apart, so that spacer 1114 is variably adjustable to more than one adjusted length. When a telescoping portion 1118 has reached the end of its axial travel in either direction, it forms a friction fit with the component that it has slid relative thereto, thereby maintaining that axially extended (or compressed) end position. Alternatively, a locking mechanism can be provided between telescoping components, one example of which is illustrated in the sectional view of FIG. 9F. In this arrangement, one of the components (a telescoping portion 1118 as shown, although it could be a fixed portion of spacer 1114 that a telescoping portion 1118 slides relative to) is provided with a groove 1120 and the component that it slides relative to (another telescoping portion 1118 or a fixed portion) is provided with a protrusion or peg 1122 that fits in groove 1120 and is slidable therein. Groove 1120 extends axially over a distance equal to the extent (distance) that the telescoping portion 1118 can slide away from the other component. At proximal and distal end of groove 1120, groove changes directions to extend in a direction perpendicular to the axially extending portion. Thus, when telescoping portion is in its most collapsed configuration, it can be rotated to place peg 1122 in a portion of groove 1120 that extends perpendicular to the axial direction, thereby preventing axial movements of telescoping portion 1118 relative to the other component. To unlock the telescoping portion 1118, it can be rotated in the opposite direction to align peg 1122 with the axially extending portion of groove 1120. Telescoping portion 1118 can then be pulled away from the other component as peg slides along the axially extending portion of groove 1120 until peg 1122 abuts against the end of the axially extending portion of groove 1120. Telescoping portion 1118 can then be rotated again in the first direction to drive peg 1122 into the portion of groove 1120 that extends perpendicular to the axial direction at the opposite end, thereby locking telescoping portion 1118 in the extended configuration. This mechanism can be provided for each telescoping portion 1118 so that each telescoping portion can be individually locked, unlocked and axially slid.

FIG. 9G illustrates another embodiment of an axially adjustable spacer 1114. In this configuration, the distal 1114a and proximal 1114b portions of spacer 1114 that include the mateable male and female tapered luer surfaces 114m and 1114f are interconnected by an adjustment member 1124. In FIG. 9G, the adjustment member 1124 comprises a screw threaded shaft that is fixed to one of the components 1114a and 114b and is threadably mated with the other component. Thus, by rotating one of the portions 1114a, 114b relative to the other, this causes adjustment member 1124 to screw into the component that it is threadably mated with or to screw out of it, depending on the direction of rotation. These actions change the overall length of spacer 1114 and thus the distance 1114p by which the spacer reduces the predetermined length of the distal tip of the stylet 100 past the distal end of irrigation catheter 10. Although not shown in FIG. 9G, adjustment member 1124 includes an axially extending lumen 1116 that communicates with lumen 1116 in the remainder of spacer 1114 so that spacer 1114 can be slid over stylet shaft 102. This embodiment can also include a slit 1116s as described above, and as illustrated in FIG. 9H, so that this embodiment of spacer 1114 can be installed without having to remove stylet 100 completely from irrigation catheter 10.

FIG. 9I illustrates an embodiment of an irrigation system in which a predetermined distance of extension of the distal tip of stylet 100 beyond a distal end of irrigation catheter 10 is adjustable. In this embodiment, an adjustment member 1124 of the type described with regard to FIGS. 9G and 9H above is built into hub 114 of stylet 100. Accordingly, rotation of the knob or proximal end portion 114b of hub 114 relative to the distal portion 114a cause the separation between these portions to either increase or decrease, depending upon the direction of rotation, and consequently either decrease or increase the predetermined distance, respectively. In this case, adjustment member 1124 need not have axially directed lumen 116 as the shaft 102 of stylet 100 can be connected at the distal portion 114a of hub 114. Hub 114 may be configured with detents, so that the mechanism “clicks” or when a separation distance between components 114a and 114b reaches a distance that may be particularly interesting to the user. For example, clicking may occur at increments of every 10 mm or some other predefined click distances. Additionally or alternatively, adjustment member 1124 and/or distal tip portion 106 may be provided with indicators markings 1126 that visually indicate to the user when a particular predetermined distance has been reached.

FIG. 9J shows a variation of the arrangement of FIG. 9I in which the adjustment member 1124 has been is built into hub 14 of irrigation catheter 10, between distal and proximal portions 14a and 14b. In this case, adjustment member 1124 has an axial lumen 116 to allow shaft 102 to pass therethrough and slide with respect to adjustment member 1124.

As noted above, the distal tip portion 106 has an atraumatic distal end or tip, which, for example, may be rounded or some other blunt configuration to prevent damage to tissues that it runs up against during insertion of the stylet. This is the same, whether distal tip portion 106 is a portion of a removable stylet 100 or is fixed at the distal end of irrigation catheter. The further characteristics described below are also applicable to both removable stylet 100 distal end portions 106 as well as distal end portions that are fixed to the irrigation catheter.

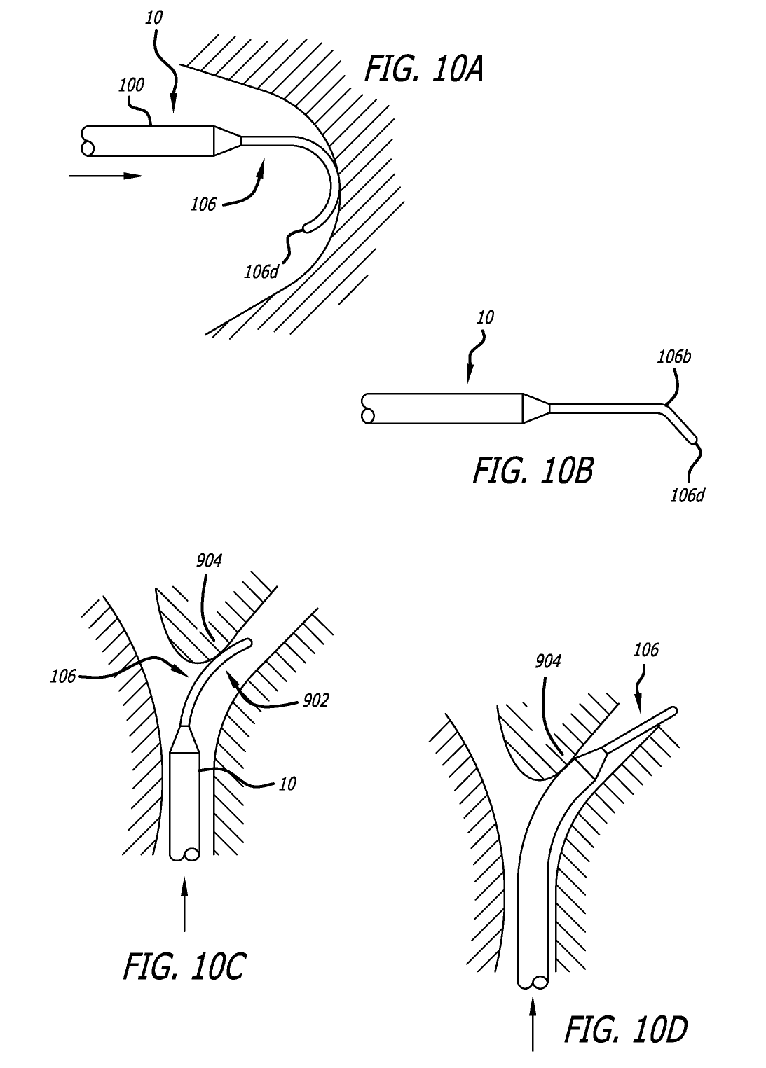

At least the distal portion of the distal tip portion 106 is flexible, so that when the tip 106d contacts and obstruction 900, the distal tip portion 106 bends over, as illustrated in FIG. 10A, rather than acting as a spear point. This flexibility also allows tip 106d to reorient at the distal tip portion 106 bends, providing it an opportunity to find a pathway where it can be further advanced, rather than simply being obstructed by the obstruction 900 with no way to change direction. Further, the bend should be a gradual bend, as shown, as the distal tip portion 106 should not kink, but bend gradually when abutting an obstruction.

At least a portion of the distal tip portion 106 that extends beyond the distal end of irrigation catheter 10 is also shapeable. The term “shapeable” is used to refer to the ability of a user to impart a bend or curve on the tip via plastic deformation, so that the curve or bend is retained in the tip portion 106 after release of bending force by the user, as illustrated in FIGS. 3F and 10B. This curve or bend 106b that is set into the distal end portion improves the ability to steer the distal end portion 106 as the irrigation system is advanced through the tortuous or branching anatomic pathways, since by rotating the system, this changes the direction in which the bent tip points and facilitates directing it in one particular direction or another.

Additionally, the distal tip portion 106 is supportive. That is, although it is sufficiently flexible to bend when directly contacting an obstruction 900, as described with regard to FIG. 10A, when it is steered into a passageway 902 and obliquely contacts a wall 904 defining the passageway 902, as illustrated in FIG. 10C, distal tip portion is sufficiently stiff to steer irrigation catheter 10 into the passageway 902, as illustrated in FIG. 10D, rather than flopping over or prolapsing when irrigation catheter 10 is pushed on from a proximal location to drive the irrigation catheter into the passageway.

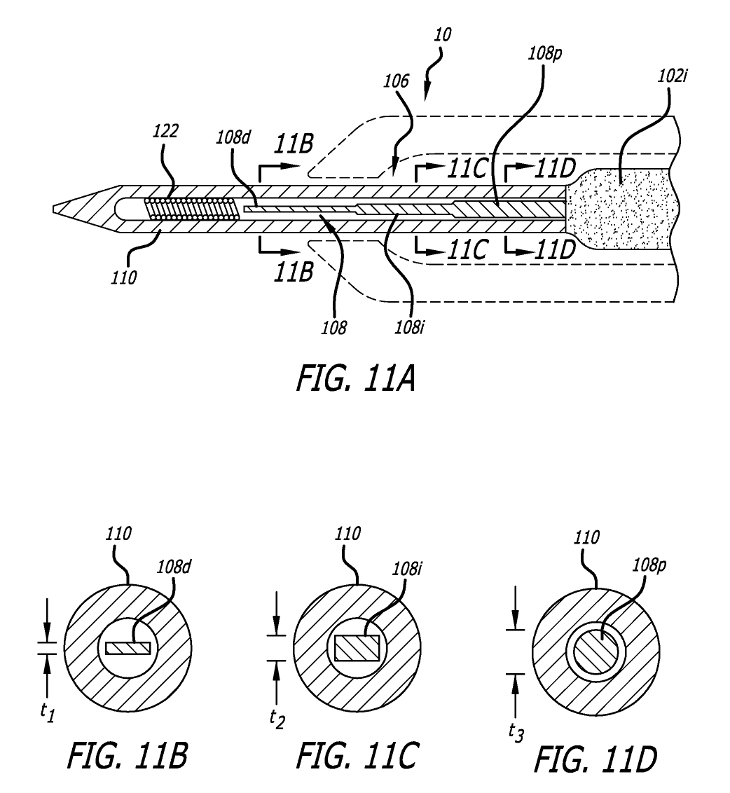

These conflicting design goals can be achieved according to the various embodiments for design of a distal tip portion 106 described herein, such as described in one embodiment with regard to FIGS. 4A-4C, for example. FIGS. 11A-11D illustrate another embodiment of a distal tip portion 106 of a stylet 100 that satisfies the flexibility, shapeability and supportive requirements described above. As already noted, this design, like others described herein is equally applicable to a distal tip portion 106 of a removable stylet, as well as a distal tip portion 106 fixed to a distal end of an irrigation catheter 10.

FIG. 11A is a longitudinal sectional view of distal tip portion 106 and a distal portion of an intermediate segment of stylet 100 joining distal tip portion, with a distal end portion of irrigation catheter 10 shown in phantom lines. In this embodiment, shaper wire 108 is provided with varying cross sections so that the bending strength increases in a direction from the distal end of shaper wire 108 to the proximal end. In the example shown, shaper wire 108 has three sections of varying cross-sectional dimension which directly varies the bending strength of each section. However, shaper wire can be formed in more or less than two sections of varying cross section. Further alternatively, shaper wire 108 can be formed with a constantly varying cross-sectional area over all or a portion of the shaper wire so that the bending strength varies constantly over such length.

In the embodiment shown, section 108d, the distal most section that extends distally of the distal end of irrigation catheter 10 has the relatively smallest cross-sectional area to provide the relatively greatest amount of flexibility and to allow a shape to be readily set. FIG. 11B shows a full cross-sectional view of distal tip portion 106 taken at a location indicated by arrows 11B-11B on the sectional view of FIG. 11A. The intermediate section 108i has a relatively larger cross-sectional diameter than the distal section 108d, as clearly shown by comparing FIGS. 11A and 11C, and a relatively smaller cross-sectional diameter than that of proximal section 108p, shown in FIG. 11E. In this embodiment distal section 108d is flattened into a ribbon having a thickness t1, intermediate section 108i is flattened into a ribbon having a thickness t2 that is greater than t1 and proximal section 108p maintains a circular cross section, with a thickness or diameter t3 greater than t2.

The proximal end of distal tip portion 106 joins the proximal end of intermediate portion 102i at a location that is inside of irrigation catheter 10 for embodiments using a removable stylet 100, as shown in FIG. 11A. This helps with supportive properties as the distal en of irrigation catheter 10 aids in providing support and reinforces the joint between distal tip portion 106 and intermediate portion 102i.

In one particular embodiment, shaper wire 108 is made from 0.008″ stainless steel wire and flattened portions 108d and 108i have thicknesses t1 of about 0.02″ and t2 greater than about 0.02″ but less than about 0.08″. The outer polymer jacket 110 comprises an 0.08″ thick wall of Pebax tubing of 40 durometer Shore hardness. Marker coil 122 is platinum or tungsten and has an outside diameter of about 0.16″, a length of about 5 mm and is formed of a coil wire having a wire diameter of about 0.02′ to about 0.03″. The intermediate (and proximal (not shown in FIG. 11A)) portions 102i and 102p are formed of Pebax beading having a Shore hardness of 72 durometer. Ribbon 108d is thin and wide for increased shapeability and flexibility in one plane.

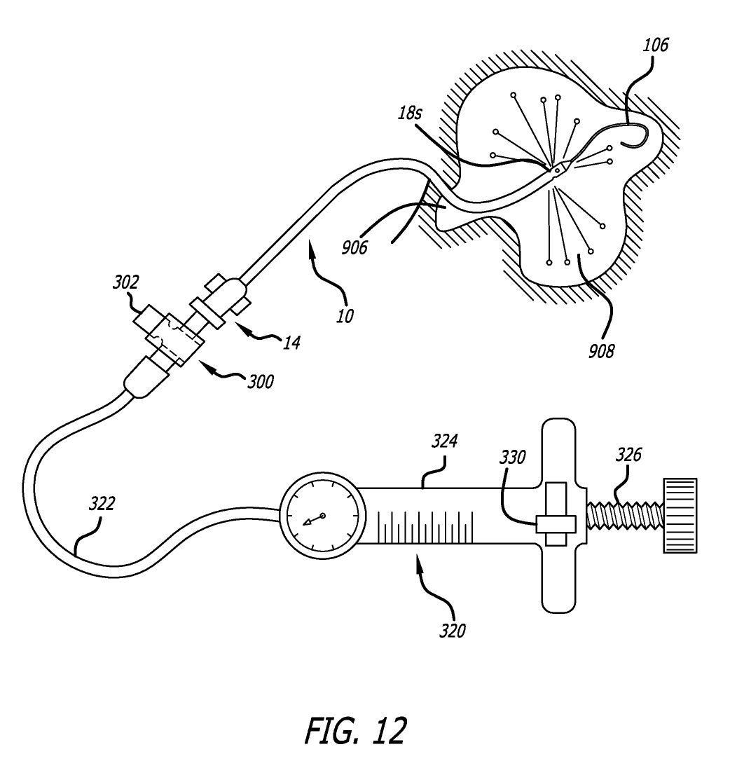

FIG. 12 illustrates an arrangement for delivering high pressure irrigation to an anatomical site within a patient. In a standard irrigation procedure, where a syringe (e.g., having a volume of about 10 cc to about 60 cc, for example) is used to drive the irrigation fluid out of the openings 18s (and optionally, 18a) by hand pressure by the operator on a hand pushable plunger of the syringe, fluid pressures of about 4 to about 6 pounds per square inch (psi) are typically generated when using a 60 cc syringe, and pressures of about 15 to about 25 psi can be generated using a 10 cc syringe. In the example shown in FIG. 12, an irrigation system comprising an irrigation catheter 10 having an integrated stylet distal tip portion 106 is used. It is noted that an irrigation system comprising an irrigation catheter 10 and removable stylet 100 could be substituted to also perform high pressure irrigation. After maneuvering the irrigation system through the tortuous anatomy, either with our without use of a guide catheter, and steering the system through sinus ostium 906 by steering the distal tip portion 106 as described above, with irrigation catheter 10 is advanced, following distal tip portion 106 into sinus 908 and delivered deep into the sinus 908 as shown in FIG. 12.

The Luer connector of hub 14 is connected to a high pressure valve 300 in fluid communication therewith, which is in turn connected to a high pressure inflation device 320 via conduit 322. As shown, high pressure inflation device 320 comprises a screw-threaded pump of a type that can be used to inflate balloon catheters, and has a screw-threaded plunger that can be torqued (or, alternatively, a rack and pinion driving mechanism can be substituted) to develop high pressure within reservoir 324 that contains an irrigation fluid (e.g., saline or other irrigating fluid used in ear, nose and throat practice), and locked via locking mechanism 330 to maintain the high pressure until it is released by opening valve 300. Other types of high pressure pumps could be substituted, including motor-driven pumps. A pressure valve 332 can be provided in fluid communication with reservoir 324 to provide feedback to the user as to how much pressure is developed in the chamber/reservoir prior to releasing the irrigation spray.

In the example shown, valve 300 is a high pressure push-button valve that is normally closed, but opens upon depressing push-button 302. Of course, other alternative types of valves can be substituted here as long as they are rated for sufficiently high pressure and are operable between closed and open states. Once the distal end portion of irrigation catheter 10, including side openings 18s, has been appropriately placed in a target where it is desired to perform the high pressure irrigation, such as deep in the sinus 908 in the example shown in FIG. 12, pressure is built up in chamber 324 by advancing plunger 326, while valve 300 remains closed. Once sufficient pressure has been established in chamber 324 (e.g., pressures of about 50 to 100 psi, or about 100 to 150 psi, or about 150 to 300 psi, or pressures greater than about 25 psi can be generated), valve 300 is opened, such as by depressing button 302 in the example of FIG. 12, and a high pressure spray is impulsively driven and jest out of side openings 18s, developing vortices of flow in directions circumferentially around tubing 12 to provide a scrubbing type of wash to the walls of the ostium. This enable a surgeon to direct a high-pressure jet inside the sinus cavity 908 to remove debris, mucus, fungus, etc.

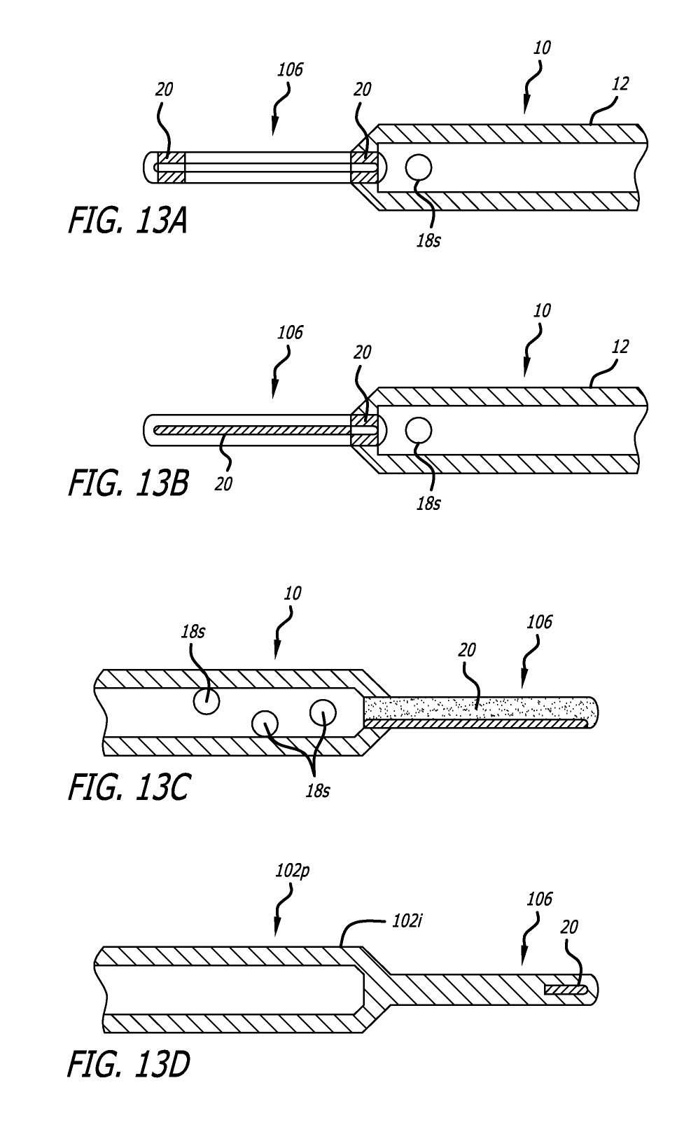

FIG. 13A illustrates another embodiment of an irrigation system in which irrigation catheter 10 has an integrated stylet distal tip portion 106. Distal tip portion 106 has radiopaque markers 20 at intermittent locations along an axial length thereof and a proximal end portion of distal tip portion is joined within the distal end portion 16 of irrigation catheter 10. In one particular embodiment according to this arrangement, the proximal end portion of distal tip portion 106 is received in irrigation catheter to a length of no greater than about three mm, distal tip portion 106 is made of 55 durometer Pebax and has an outside diameter of about 0.35″, radiopaque markers 20 are platinum coils made from platinum wire with the coils having a length each of about 2 mm and four side holes each having a diameter of about 0.50″ are punched in catheter tubing 12 adjacent the distal end of irrigation catheter 10, wherein tubing 12 is made of 55 durometer Pebax having a wall thickness of about 0.13″. In an embodiment having a distal tip portion 106 about 20 mm long, two platinum coils 20 are placed, as shown in FIG. 13A. For an embodiment having a distal tip portion 106 about 40 mm long, three platinum coils 30 are spaced substantially equidistantly apart in the shaft of distal tip portion 106. In an alternative embodiment, distal tip portion 106 is formed of 55 durometer, barium sulfate-filled Pebax and a platinum coil 20 having a length of about 8 to 10 mm long in placed in the distal tip portion 106. Alternatively, markers 20 can be made of stainless steel, or tungsten, or of any of the other materials mentioned previously.

FIG. 13B illustrates an embodiment in which distal tip portion 106 is about 30 mm long. A polymer inner core is provided (e.g., solid Pebax, 55 durometer having a diameter of about 0.20″ to about 0.22″) and a metallic radiopaque coil marker 20 is wrapped around the inner core. In one particular embodiment, the marker is a stainless steel coil having a diameter of about 0.22″. In another particular embodiment, the marker is a tungsten coil having a diameter of about 0.22″ to 0.24″. An outer layer of Pebax encapsulates the core and marker 20 to give the distal tip portion an outside diameter of about 0.35″. An additional radiopaque marker 20 is provided in the proximal end portion of distal tip portion 106 in the form of a marker band, in this case a platinum marker band having an outside diameter of about 0.30″ to about 0.36″.

FIG. 13C illustrates an embodiment having similar tubing 12 to previously described embodiments, and having a distal tip portion 106 the extends form the distal end of irrigation catheter 10 by a length of about 12 mm. Distal tip portion 106 is soft and shapeable, and made of 55 durometer Pebax, with a platinum marker coil 20 extending about 10 mm from the distal end of irrigation catheter 10. The Pebax polymer of distal tip portion 106 is melted over the marker coil during production, so that marker coil 20 is engulfed by the polymer material. Axially adjacent side holes 18s in this embodiment are separated by an axial distance of about 7 mm, and three side holes 18s are provided, each having a diameter of about 0.50″. It is again noted here that these specification are for one particular embodiment, and that the arrangement shown in FIG. 13C is not limited to these specifications, as the specifications may vary, for example, as noted by the remainder of the disclosure herein, or by other equivalent variations.

FIG. 13D illustrates an embodiment where distal tip portion 106 includes a solid polymer tip that contains a radiopaque coil 20 at a distal portion of the distal tip portion 106.

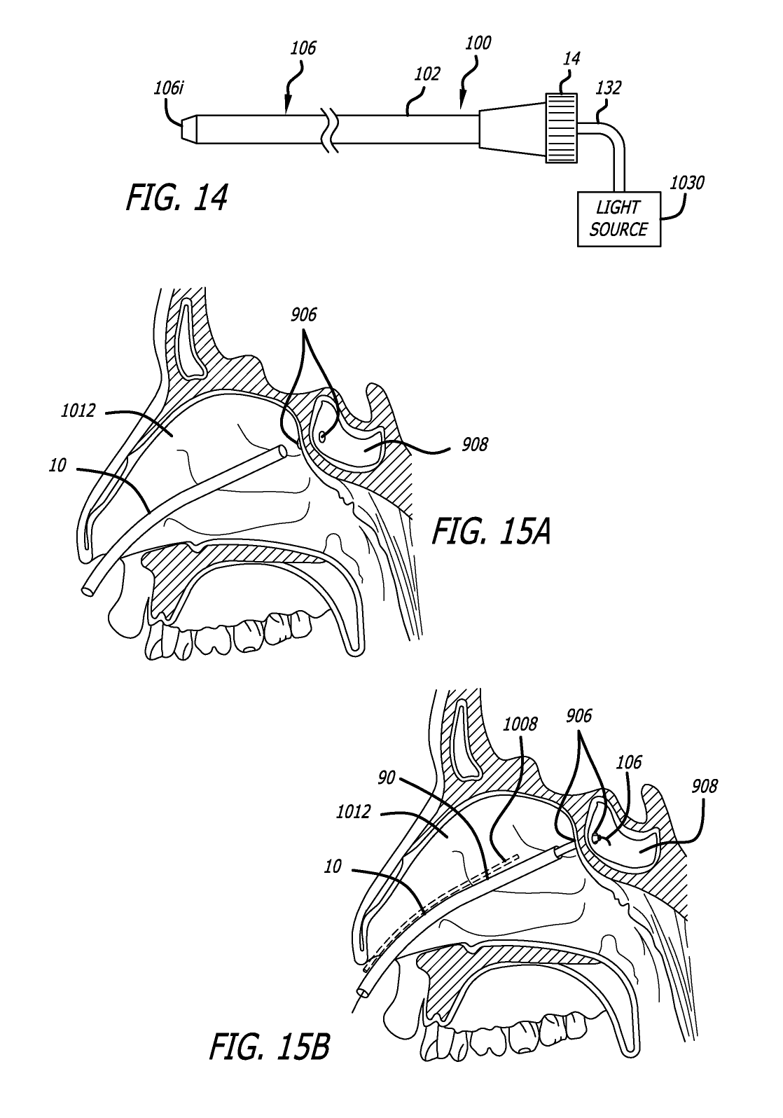

FIG. 14 illustrates a removable illuminating stylet 100 that includes an illuminating distal tip 106i at a distal end of the distal tip portion 106. One or more illumination channels 10i are provided in stylet 100 and extend the length thereof. Illumination channels 10i are configured to transport light from the proximal end of stylet 100 to and out of the distal end 106i. In the example shown, two illumination channels are provided, each comprising a plastic illumination fiber. The plastic used to make the illumination fibers is compounded for light transmission properties according to techniques known and available in the art. As one example, ESKA™ (Mitsubishi Rayon), a high performance plastic optical fiber may be used, which has a concentric double-layer structure with high-purity polymethyl methacrylate (PMMA) core and a thin layer of specially selected transparent fluorine polymer cladding.

Alternatively, a single plastic illumination fiber 10i may be used, or glass illumination fibers may be substituted which are much smaller in outside diameter, e.g., about 0.002″. In this case, more illumination fibers may be provided in a bundle.

The distal end of stylet 100 can be sealed by a transparent (or translucent) seal 106i which may be in the form of epoxy or other transparent or translucent adhesive or sealing material. Seal 106i maintains the distal ends of illumination fibers 10i coincident with the distal end of stylet 100 and also provides an atraumatic tip of the device 100. Further, seal 106i prevents entrance of foreign materials into the device. The distal end can be designed to either focus or distribute the light as it emanates therefrom, to achieve maximum transillumination effects. In this regard, the distal end can include a lens, prism or diffracting element.

The proximal end of tubing 102 can also be sealed by a transparent (or translucent) seal which may be in the form of epoxy or other transparent or translucent adhesive or sealing material. This proximal end seal maintains the proximal ends of illumination fibers 10i coincident with the proximal end of stylet tubing 102. The proximal end of device 10 maybe further prepared by grinding and polishing to improve the optical properties at the interface of the proximal end of stylet 100 with a light source.

A light source 1030 is connected to stylet 100 via hub 14 in this case which is also configured as an optical connector. The couplet of hub 14 is connected to light source 1030, such as a conventional endoscope light source, for example, or other light source capable of delivering preferably at least 10,000 lux through hub 14. Light cable 1032 optically connects the connector of hub 14 100 with light source 1030 to deliver light from the light source 1030 to hub 14. Light cable 1032 can optionally be a fluid-filled light cable, such as the type provided with DYMAX BlueWave™ 200 and ADAC Systems Cure Spot™ light cables, for example. A liquid filled light cable comprises a light conducting liquid core within plastic tubing. The liquid is non-toxic, non-flammable and transparent from 270 to 720 nm. The ends of a liquid filled light cable can be sealed with high quality quartz glass and metal spiral tubing surrounded by a plastic sleeve for exterior protection.

Light transmitted to hub 14 via light cable 1032 is directed through illumination channels 10i and delivered out of tip 106i to provide an illumination at the distal tip of stylet 100. By providing stylet 100 with this light emitting capability, the distal tip 106i when illuminated during traversing the stylet 100 and irrigation catheter 10 through the tortuous anatomy causes a process known as transillumination to occurs. Thus, as the irrigation system traverses through the sinus passageways, passes through an ostium and enters a sinus cavity, transillumination, which is the passing of light through the walls of a body part or organ shows a light spot on an external surface of the patient. Thus, when distal tip 106i of stylet 100 is located in a sinus, the light emitted from tip 106i passes through the facial structures and appears as a glowing region on the skin (e.g., face) of the patient. As the tip 106i gets closer to the surface of the structure that it is inserted into (e.g., the surface or interior wall of the sinus), the transillumination effect becomes brighter and more focused (i.e., smaller in area). Additionally, the movements of the stylet tip 106i can be tracked by following the movements of the transillumination spot produced on the skin of the patient.

Further details about the components for making a stylet as an illuminating stylet are described in co-pending, commonly assigned application Ser. No. 11/522,497 filed Sep. 15, 2006 and titled Methods and Devices for Facilitation Visualization in a Surgical Environment” which is hereby incorporated herein, in its entirety, by reference thereto.

Illuminating stylet 100 may be provided with any of the features described above with regard to other embodiments of stylet 100 and with regard to distal tip portion 106.

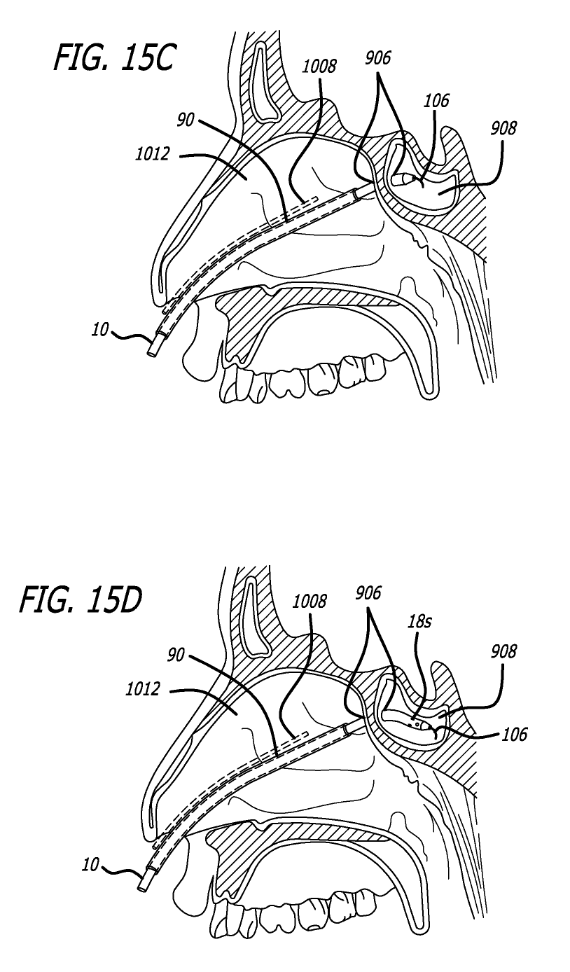

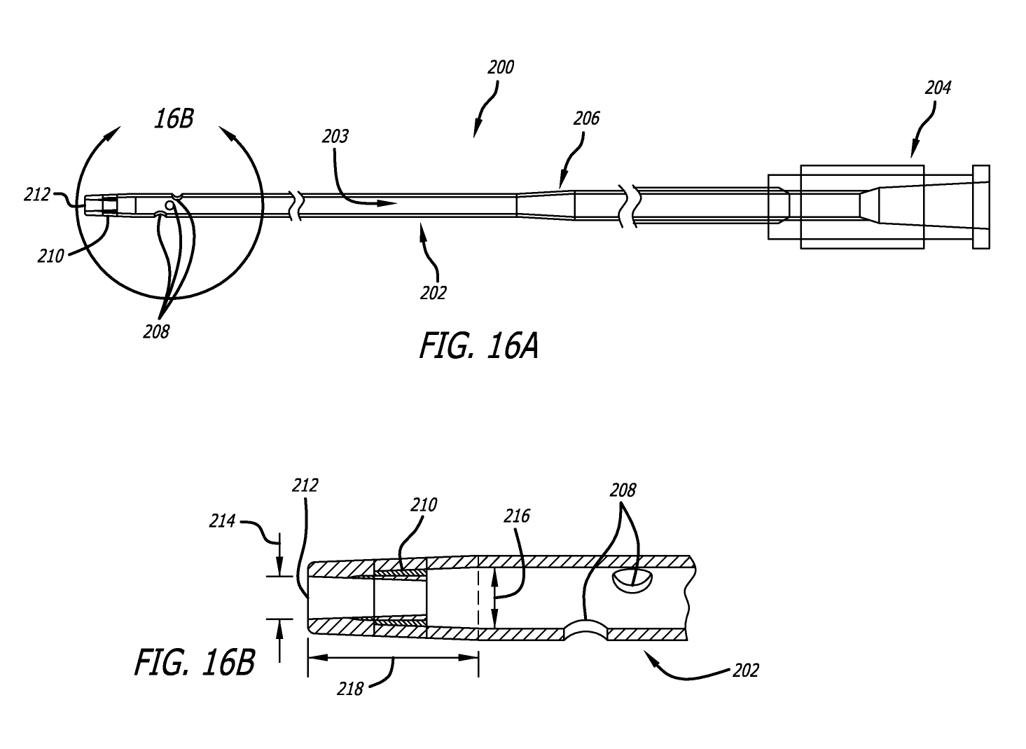

FIGS. 15A-15D are illustrations of partial sagittal sectional views through a human head showing various steps of a method of gaining access to a paranasal sinus by an irrigation system as described herein to perform at least one of irrigation, suction, delivery of a therapeutic or diagnostic substance or retrieval of a culture. In FIG. 15A, a first introducing device in the form of a sinus guide 90 is introduced through a nostril and through a nasal cavity 1012 to a location close to an ostium of a sphenoid sinus 908. It is noted that this step is optional for placement of an irrigation system of a type described herein and may not be needed for accessing certain spaces in the anatomy, including sphenoid sinuses. Alternatively, irrigation system can be navigated without the use of sinus guide 90, by steering the tip of stylet distal end portion 106 to steer distal end portion 106 and the distal end portion of irrigation catheter 10 though ostium 906 and into sinus 908. For other harder to reach anatomical locations, such as the maxillary sinus, for example, a sinus guide 90 is typically used. For accessing a maxillary sinus, a sinus guide having a bend of 110 degrees may be needed.

Sinus guide 90 may be straight, malleable, or it may incorporate one or more preformed curves or bends as further described in U.S. Patent Publication Nos. 2006/004323; 2006/0063973; and 2006/0095066, for example, each of which are incorporated herein, in their entireties, by reference thereto. In embodiments where sinus guide 90 is curved or bent, the deflection angle of the curve or bend may be in the range of up to about 135 degrees, and irrigation catheter 10 does not kink as it is passed through this bent portion of the guide catheter.

In FIG. 15B, an irrigation system comprising an irrigation catheter 10 and stylet distal end portion 106 are introduced through the first introduction device (i.e., sinus guide 90) and advanced so that the stylet distal end portion 106 exits the guide catheter 90. Stylet distal end portion is then steered into the sinus ostium 906 and pushed into the sinus 908 with the irrigation catheter following due to the supportive characteristics of stylet distal end portion 106. The irrigation system may include an irrigation catheter 10 with integrated stylet distal end portion 106 or irrigation catheter 10 with removable stylet 100. In either case, the irrigation system can alternatively be steered and delivered from the entry at the nostril through the tortuous anatomy including the ostium and into the sinus without the use of the guide catheter 90, as noted. In any case, if the surgeon decides that the predetermined length of the stylet distal end 106 is not optimal, the irrigation system can be withdraw to clip a portion of the stylet distal end 106, after which the irrigation system is reintroduced to continue the procedure, or the surgeon can adjust the predetermined length of the stylet distal end 106 to either shorten or lengthen it, using any of the features at the proximal end of the system that were described above.

In FIG. 15C, the distal end of irrigation catheter has entered the sinus by steering the stylet distal end portion 106 and pushing on a proximal end portion of the irrigation catheter 10 or irrigation catheter 10 and mated stylet 100. Thereafter, in FIG. 15D, the distal end of irrigation catheter 10 is inserted deep into sinus 906, so that side openings 18s are positioned well within the sinus cavity to perform at least one of the functions noted above. Further, two functions, such as simultaneous irrigation and suction may be performed with at least some of the irrigation systems described herein. FIG. 15D illustrates an irrigation procedure being performed, as irrigation fluid is jetted through openings 18s to establish vortices to clean the walls of the sinus by the irrigation flow.

These procedures may be performed as stand-alone procedures, or they may be follow-up procedures performed after performing some other procedure such as a dilation of the ostium, as just one example. In this case, when a guidewire is used to deliver a working tool to the anatomy through guide catheter 90, for example, the working tool and guidewire are both removed prior to insertion of the irrigation system as described above. Further alternatively, the working tool can be removed while leaving the guidewire in place, and an irrigation catheter 10 that is designed to function with a removable stylet 100 can instead be delivered over the guidewire without the use of the removable stylet 100. In uses where irrigation system includes an irrigation catheter 10 delivered with a mated removable stylet 100, the stylet 100 is removed prior to performing an irrigation, suction, substance delivery or culture retrieval function. Similarly, where irrigation catheter 10 is delivered over a guidewire, the guidewire is removed prior to performing an irrigation, suction, substance delivery or culture retrieval function. Suction may be provided with a syringe or with an operating room suction source, for example.

It is further noted that irrigation systems described herein are not limited to only being delivered through a natural anatomic pathway, but can also be delivered though a surgical opening to irrigate, suction, deliver therapeutic and/or diagnostic substances and/or take cultures. For example, a hole may be trephined to provide direct access to the frontal sinus and an irrigation system as described herein can be delivered through the hole to flush a frontal sinus. This technique can be particularly useful for a sinus that does not communicate normally with the middle meatus. As another example, the anterior wall of the ethmoid bulla can be punctured and an irrigation system as described herein can be inserted therethrough to flush the anterior ethmoid sinuses. This procedure may be done after removal of an ethmoid sinus stent, for example. An irrigation system described herein can be delivered though a maxillary antrosotmy of Caldwell-Luc incision to perform any of the above described functions. Still further, an irrigation system as described herein may be delivered through the Eustachian tube or an incision to access the middle ear to perform any of the above-described functions in the location of the middle ear.

FIGS. 15B-15D show an optional scope 1008 in dotted lines, that may be inserted to provide visualization of advancement of sinus guide 90 and/or inserted alongside sinus guide 90 or integrated with sinus guide 90, such as described in U.S. Patent Application Publication 2006/0063973, for example, which is hereby incorporated herein in its entirety, by reference thereto. Scope 1008 may be a flexible scope. It is to be appreciated that optional scope 1008 may comprise any suitable types of rigid or flexible endoscope and such optional scope may be separate from or incorporated into the sinus guide 90 or irrigation catheter 10. Further information about such endoscopes can be found in co-pending provisional Application Ser. No. 60/844,874, filed Sep. 15, 2006 and titled “Endoscopic Methods and Devices for Transnasal Procedures, which is hereby incorporated herein, in its entirety, by reference thereto.

Scope 1008 may provide visualization of insertion of the guide catheter 90 and or at least partial visualization of advancement of the irrigation system when inserted with or without guide 90. An illuminating stylet may be used in combination with scope 1008 to provide enhanced visualization as described in more detail in application Ser. No. 11/522,497.

Delivery of an irrigation system, with or without use of a guide catheter may be additionally or alternatively visualized by fluoroscopy, electromagnetic or optical guidance, including 3-dimensional visualization such as CT or MRI visualization or other known visualization techniques.

In most of the embodiments described above, an irrigation catheter may be delivered into a sinus over a guidewire rather than over a stylet. (An exception includes the embodiment depicted in FIGS. 5A-5C, which has a non-removable sylet). To deliver an irrigation catheter over a guidewire, for example, a distal end of the guidewire may first be advanced into a sinus through a guide catheter, as described above and in a number of the patent applications previously incorporated by reference. In one embodiment, placement of a guidewire in a desired position in a sinus may be confirmed by fluoroscopy. Once the guidewire is in position, a flexible irrigation catheter such as many of the embodiments described above may be advanced over the guidewire to position a distal portion of the catheter in the sinus. In various embodiments, the irrigation catheter may be advanced over the guidewire through a guide catheter or, alternatively, over the guidewire without using a guide catheter. Positioning of a distal portion of the irrigation catheter in the desired sinus may be confirmed using fluoroscopy, in embodiments where the irrigation catheter distal portion includes a radiopaque marker or material. In some embodiments, the guidewire may then be withdrawn through the irrigation catheter. Irrigation fluid may then be introduced through the irrigation catheter, and in some embodiments fluid may be suctioned back through the irrigation catheter. In various embodiments, any other suitable guidewire-based delivery techniques may be employed to advance an irrigation catheter of the present invention.

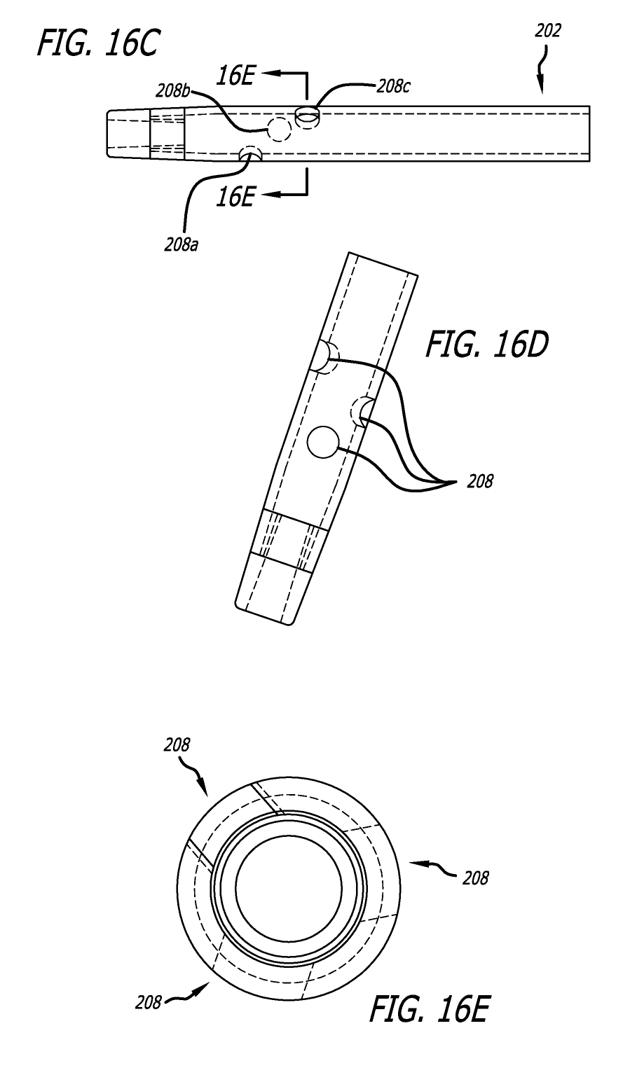

Referring now to FIGS. 16A-16E, one embodiment of a flexible irrigation catheter 210 which may be advanced over a guidewire (not shown) may include a catheter body 202 having a lumen 203, a proximal hub 204, a strain relief portion 206, multiple irrigation side ports 208, a radiopaque marker 210 and a distal irrigation port 212. Side ports 208 and distal port 212 are in fluid communication with lumen 203. In various embodiments, catheter 210 may have any suitable dimensions and may be made of any suitable materials to facilitate advancement into and irrigation of one or more paranasal sinuses. In one embodiment, for example, catheter body 202 may be made of PEBAX, strain relief portion 206 may be made of PEBAX covered in heat-shrink polyolefin, and radiopaque marker may be made of titanium, iridium or a combination thereof. Of course, in other embodiments one or more alternative materials may be used to make one or more of these components.

Referring to FIG. 16A, catheter body 202 may have any suitable length for treating one or more of the paranasal sinuses. In one embodiment, for example, catheter body 202 may have a length of between about 10 inches and about 20 inches and more preferably between about 12 inches and about 18 inches and even more preferably between about 13 inches and about 15 inches. In one embodiment, strain relief portion 206 may be located back from the distal end of catheter body 202 between about 4 inches and about 6 inches and more preferably about 5 inches. In various embodiments, the outer diameter of catheter body may be between about 0.050 inches and about 0.100 inches and more preferably between about 0.070 inches and about 0.080 inches.

With reference to FIG. 16B, in some embodiments, catheter body 202 may include a distal tapered portion 218. Tapered portion 218 may measure, for example, between about 0.100 inches and about 0.200 inches in some embodiments, and more preferably between about 0.130 inches and about 0.170 inches and even more preferably between about 0.140 inches and about 0.160 inches. An inner diameter 214 of distal port 212 may measure between about 0.020 inches and about 0.050 inches and more preferably between about 0.030 inches and about 0.040 inches. An inner diameter 216 of catheter body 202 proximal of tapered portion 218 may measure between about 0.030 inches and about 0.070 inches and more preferably between about 0.040 inches and about 0.060 inches.

Referring now to FIG. 16C, in one embodiment, irrigation catheter 210 may include three side ports 208a, 208b, 208c, distributed along catheter body 202 in a helical pattern. Of course, alternative embodiments may include any suitable alternative number of side ports distributed in any suitable pattern. In one embodiment, a first side port 208a may be placed at about 5 mm±0.5 mm from the distal end of catheter body 202, a second side port 208b may be placed at about 6 mm±0.5 mm from the distal end of catheter body 202, and a third side port 208c may be places at about 7 mm±0.5 mm from the distal end of catheter body 202, with each of these measurements being from the distal end to approximately the center of each side port 208.

Referring to FIG. 16D, side ports 208 may have any suitable diameter in various alternative embodiments. For example, in one embodiment, each side port 208 may have a diameter of between about 0.020 inches and about 0.050 inches and more preferably between about 0.030 inches and about 0.040 inches and even more preferably about 0.033 inches±0.003 inches.

With reference to FIG. 16E, which depicts the cross-section E-E from FIG. 16C, in one embodiment side ports 208 may be distributed around the circumference of catheter body 202 approximately 120° apart from approximately the center of each port 208 to the center of each subsequent port 208.

The embodiment of flexible irrigation catheter 210 just described is one exemplary embodiment, and any of a number of changes may be made to the design of irrigation catheter 210 in alternative embodiments. In some embodiments, catheter 210 may be suitable for advancement over a guidewire, in some embodiments it may be suitable for use with a stylet, and in some embodiments it may be suitable for use with either a guidewire or a stylet.

While the present invention has been described with reference to the specific embodiments thereof, it should be understood by those skilled in the art that various changes may be made and equivalents may be substituted without departing from the true spirit and scope of the invention. In addition, many modifications may be made to adapt a particular situation, material, composition of matter, process, process step or steps, to the objective, spirit and scope of the present invention.

We claim:

1. A method for irrigating a paranasal sinus, the method comprising:
advancing a distal portion of a sinus irrigation catheter through an ostium of a paranasal sinus into the sinus; and
passing fluid through multiple ports disposed at different locations along a length of the catheter distal portion into the sinus.

2. The method of claim 1, wherein advancing the distal portion comprises advancing over a guidewire.

3. The method of claim 2, further comprising removing the guidewire through the irrigation catheter before passing the fluid.

4. The method of claim 3, wherein passing the fluid comprises passing fluid through three side ports distributed in a helical pattern along a length of the irrigation catheter and one distal port on the irrigation catheter.

5. The method of claim 1, wherein advancing the distal portion comprises advancing over a stylet.

6. The method of claim 1, wherein passing the fluid comprises passing fluid through multiple side ports and one distal end port on the irrigation catheter.

7. The method of claim 6, wherein passing the fluid comprises forming a vortex of fluid in the sinus.

8. The method of claim 6, wherein passing the fluid comprises passing fluid through three side ports distributed in a helical pattern along a length of the irrigation catheter and one distal port on the irrigation catheter.

9. The method of claim 1, further comprising:
bending the distal portion before advancing it into the sinus; and
turning the irrigation catheter while advancing it to steer the bent distal portion toward the sinus ostium.

10. The method of claim 1, further comprising suctioning at least some of the fluid back into the irrigation catheter through at least one of the ports.

11. The method of claim 1, further comprising suctioning at least some of the fluid out of the sinus using a separate suction device.

12. The method of claim 1, further comprising viewing a radiopaque marker of the irrigation catheter within the sinus using fluoroscopy.

13. A method for irrigating a paranasal sinus, the method comprising:
advancing a distal portion of a sinus irrigation catheter through an ostium of a paranasal sinus into the sinus; and
passing fluid through a distal end port disposed on the distal end of the irrigation catheter and three side ports disposed in a helical pattern distributed in a helical pattern along a length of the irrigation catheter into the sinus to form a vortex of irrigation fluid in the sinus.

14. A flexible irrigation catheter device for irrigating a paranasal sinus, the device comprising:
an elongate catheter body having a proximal end, a distal end, a lumen therebetween, and a tapered distal portion extending proximally from the distal end of the catheter body, wherein at least a portion of the catheter body is sized to pass through a nasal cavity and through a sinus ostium to enter a paranasal sinus;
a proximal hub coupled with the proximal end of the catheter body for connecting to a source of irrigation fluid;
multiple side ports disposed along the catheter body closer to the distal end than the proximal end and in fluid communication with the lumen; and
a distal end port at the distal end of the catheter body.

15. The device of claim 14, wherein the tapered distal portion of the catheter body measures between 0.140 inches and 0.160 inches.

16. The device of claim 15, wherein the side ports are located proximal to the tapered distal portion.

17. The device of claim 14, wherein the side ports comprise three side ports disposed in a helical pattern along the catheter body.

18. The device of claim 17, wherein the three side ports are located 5 mm±0.5 mm, 6 mm±0.5 mm and 7 mm±0.5 mm from the distal end of the catheter body.

19. The device of claim 18, wherein each of the side ports has a diameter of 0.033 inches±0.003 inches.

20. The device of claim 19, wherein each of the three side ports is disposed approximately 120° around a circumference of the catheter body relative to the other two side ports.

21. The device of claim 14, further comprising a radiopaque marker coupled with the catheter body at or near the tapered distal portion.

22. The device of claim 14, wherein a distal portion of the irrigation catheter is malleable.

23. A flexible irrigation catheter device for irrigating a paranasal sinus, the device comprising:
an elongate catheter body having a proximal end, a distal end, a lumen therebetween, and a tapered distal portion extending between 0.140 inches and 0.160 inches proximally from the distal end of the catheter body, wherein at least a portion of the catheter body is sized to pass through a nasal cavity and through a sinus ostium to enter a paranasal sinus;
a proximal hub coupled with the proximal end of the catheter body for connecting to a source of irrigation fluid;
three side ports disposed in a helical pattern along the catheter body proximal to the tapered distal portion and in fluid communication with the lumen, wherein the three side ports are located 5 mm±0.5 mm, 6 mm±0.5 mm and 7 mm±0.5 mm from the distal end of the catheter body, wherein each of the side ports has a diameter of 0.033 inches±0.003 inches, and wherein each of the three side ports is disposed approximately 120° around a circumference of the catheter body relative to the other two side ports;
a distal end port at the distal end of the catheter body; and
a radiopaque marker coupled with the tapered distal portion of the catheter body.

24. A system for irrigating a paranasal sinus, the system comprising:
an irrigation catheter comprising:
an elongate catheter body having a proximal end, a distal end, a lumen therebetween, and a tapered distal portion extending proximally from the distal end of the catheter body, wherein at least a portion of the catheter body is sized to pass through a nasal cavity and through a sinus ostium to enter a paranasal sinus;
a proximal hub coupled with the proximal end of the catheter body for connecting to a source of irrigation fluid;
multiple side ports disposed along the catheter body closer to the distal end than the proximal end and in fluid communication with the lumen; and
a distal end port; and
a guidwire over which the irrigation catheter is passable into the sinus.

25. A system as in claim 24, wherein the side ports comprise three side ports disposed in a helical pattern along the catheter body proximal to the tapered distal portion and in fluid communication with the lumen, wherein the three side ports are located 5 mm±0.5 mm, 6 mm±0.5 mm and 7 mm±0.5 mm from the distal end of the catheter body, wherein each of the side ports has a diameter of 0.033 inches±0.003 inches, and wherein each of the three side ports is disposed approximately 120° around a circumference of the catheter body relative to the other two side ports.
